(19) United States
(12) Patent Application Publication
(10) Pub. No.: US 2025/0325709 A1
(43) Pub. Date: Oct. 23, 2025

(54) GENE DELIVERY TARGETING NEURAL STEM CELLS AND NEURAL PROGENITOR CELLS

(71) Applicant: Rutgers, The State University of New Jersey, New Brunswick, NJ (US)

(72) Inventors: Li Cai, Warren, NJ (US); Zachary Finkel, Monroe Township, NJ (US); Sonia Gulati, New York, NY (US)

(73) Assignee: Rutgers, The State University of New Jersey, New Brunswick, NJ (US)

(21) Appl. No.: 19/183,437

(22) Filed: Apr. 18, 2025

Related U.S. Application Data
(60) Provisional application No. 63/636,458, filed on Apr. 19, 2024.

(51) Int. Cl. A61K 48/00; C12N 15/86

(52) U.S. Cl. CPC A61K 48/0058; C12N 15/86; C12N 2750/14143; C12N 2820/007

(57) ABSTRACT

Methods of expressing a heterologous nucleic acid in neural stem and progenitor cells (NSPCs), comprising transducing NPSCs with an adeno-associated virus 6 (AAV6) vector comprising the heterologous nucleic acid are provided. Methods of treating a neurological disorder in a mammalian subject, comprising administering to the subject a therapeutically effective amount of an adeno-associated virus 6 (AAV6) vector comprising a heterologous nucleic acid molecule, such as Gsx1, are also provided.

### CROSS REFERENCE TO RELATED APPLICATION

This claims the benefit of U.S. Provisional Application No. 63/636,458, filed Apr. 19, 2024, which is incorporated herein by reference in its entirety.

### ACKNOWLEDGMENT OF GOVERNMENT SUPPORT

This invention was made with government support under grant numbers GM008339, GM138296 awarded by the National Institutes of Health and grant number EEC1950509 awarded by the National Science Foundation. The government has certain rights in the invention.

### FIELD

This disclosure relates to compositions and methods for delivery and expression of heterologous nucleic acids in neural stem cells and neural progenitor cells.

### ELECTRONIC SEQUENCE LISTING INCORPORATION

The Sequence Listing is submitted as an XML file in the form of the file named “7213-111958-02_Sequence_Listing” (23,498 bytes), which was created on Apr. 18, 2025, which is incorporated by reference herein.

### SUMMARY

Provided herein are methods of expressing a heterologous nucleic acid in neural stem and progenitor cells (NSPCs), comprising transducing NPSCs with an adeno-associated virus 6 (AAV6) vector comprising the heterologous nucleic acid. In some aspects, the NPSCs are spinal cord NPSCs and/or the NPSCs are in an injured or disease state. In one non-limiting example, the heterologous nucleic acid encodes Gsx1.

Also provided are methods of treating a neurological disorder in a mammalian subject, comprising administering to the subject a therapeutically effective amount of an adeno-associated virus 6 (AAV6) vector comprising a heterologous nucleic acid molecule, such as a heterologous nucleic acid encoding a therapeutically effective protein for treating the neurological disorder. In some aspects, the neurological disorder is a spinal cord injury, a brain injury, Parkinson's disease, Huntington's disease, Alzheimer's disease, retinal degenerative disease or injury, or amyotrophic lateral sclerosis. In one non-limiting example, the heterologous nucleic acid encodes Gsx1.

The foregoing and other features of the disclosure will become more apparent from the following detailed description, which proceeds with reference to the accompanying figures.

### BRIEF DESCRIPTION OF THE DRAWINGS

FIGS. 1A-1B show that AAV serotype 6 targets NSPCs in acute SCI. Virally transduced cells in longitudinal spinal cord sections were analyzed at 4 dpi. FIG. 1A shows percentage of GFP+ cells over DAPI+ total cells adjacent to the lesion epicenter. FIG. 1B shows percentage of GFP+Nestin+ cells over total GFP+ cells adjacent to the lesion epicenter. Data are expressed as mean±SEM. *p<0.05, **p<0.01, AAV5-GFP, AAV6-GFP, and AAVrh10-GFP versus the control group (LV-GFP). Statistical analysis was performed using a one-way ANOVA followed by Tukey's post hoc test.

FIGS. 2A-2E illustrate that Gsx1 promotes NSPC activation, proliferation, and neurogenesis in acute hemisection and contusion SCI. For FIGS. 2A-2C, virally transduced cells were analyzed in longitudinal spinal cord sections at 4 dpi. FIG. 2A shows percentage of GFP+Nestin+ cells over total GFP+ cells adjacent to the lesion epicenter. FIG. 2B shows percentage of GFP+PCNA+ cells over total GFP+ cells adjacent to the lesion epicenter. FIG. 2C shows percentage of Nestin+PCNA+GFP+ cells over total GFP+ cells adjacent to the lesion epicenter. For FIGS. 2D-2E, virally infected (Gsx1+) proliferating (PCNA+) neural stem cells (Nestin+) in the injured spinal cord with AAV6-Gsx1 treatment were analyzed. FIG. 2D shows percentage of GFP+Nestin+ cells over total DAPI+ cells at injection sites adjacent to the lesion epicenter. FIG. 2E shows percentage of Nestin+PCNA+GFP+ cells over total DAPI+ cells adjacent to the lesion epicenter. AAV6-Gsx1 and LV-Gsx1 induce neurogenesis in the injured spinal cord. Data are expressed as mean±SEM. *p<0.05, **p<0.01, AAV6-Gsx1 and LV-Gsx1-GFP versus the control groups (AAV6-GFP, LV-GFP). Statistical analysis was performed using a one-way ANOVA followed by Tukey's post hoc test.

FIGS. 3A-3C illustrate that Gsx1 promotes neuroblast and immature neuron formation in subacute SCI. FOR FIGS. 3A-3B, virally transduced cells, immature neurons (Tuj1), and canonical notch activity (Notch1) were analyzed in longitudinal spinal cord sections at 14 dpi. FIG. 3A shows percent of Tuj1+ cells adjacent to the lesion epicenter. FIG. 3B shows percent of Notch1+ cells adjacent to the lesion epicenter. For FIG. 3C, virally transduced cells and neuroblasts (DCX) were analyzed in longitudinal spinal cord sections at 14 dpi. FIG. 3C shows percent of DCX+ cells adjacent to the lesion epicenter. Data are expressed as mean±SEM. *p<0.05, **p<0.01, AAV6-Gsx1 and LV-Gsx1-GFP versus the group (AAV6-GFP). Statistical analysis was performed using a one-way ANOVA followed by Tukey's post hoc test.

FIGS. 4A-4C illustrates that Gsx1 increases excitatory and reduces inhibitory interneuron populations in chronic SCI. For FIG. 4A, virally transduced cells (GFP) and excitatory interneurons (VGlut2) were analyzed in longitudinal spinal cord sections at 56 dpi. FIG. 4A shows percent of VGlut2+ cells adjacent to the lesion epicenter. For FIGS. 4B-4C, virally transduced cells (GFP), inhibitory interneurons (GABA) and cholinergic interneurons (ChAT) were analyzed in longitudinal spinal cord sections at 56 dpi. FIG. 4B shows percent of GABA+ cells adjacent to the lesion epicenter. FIG. 4C shows percent of ChAT+ cells adjacent to the lesion epicenter. Data are expressed as mean±SEM. *p<0.05, **p<0.01, AAV6-Gsx1 and LV-Gsx1-GFP versus the group (AAV6-GFP). Statistical analysis was performed using a one-way ANOVA followed by Tukey's post hoc test.

FIGS. 5A-5C show that Gsx1 reduces reactive gliosis and glial scar formation in subacute and chronic SCI. For FIG. 5A, virally transduced cells (GFP) and astrocytes (GFAP) were analyzed in longitudinal spinal cord sections at 14 dpi. FIG. 5A shows percent of GFAP+ cells adjacent to the lesion epicenter. For FIG. 5B, astrocytes (GFAP) and gene therapy (Virus) were analyzed in coronal spinal cord sections at 56 dpi. FIG. 5B shows percent of GFAP+ cells in the glial scar border adjacent to the lesion epicenter. For FIG. 5C, virally transduced cells (GFP) and chondroitin sulfate proteoglycans (CSPG) (CS56) were analyzed in longitudinal spinal cord sections taken at the lesion edge at 56 dpi. FIG. 5C shows percent of CS56+ signal in the scar border adjacent to the lesion epicenter. Data are expressed as mean±SEM. *p<0.05, **p<0.01, SCI+AAV6-Gsx1 and SCI+LV-Gsx1-GFP versus the control groups (SCI+AAV6-GFP, Sham+AAV6-GFP). Statistical analysis was performed using a one-way ANOVA followed by Tukey's post hoc test.

FIGS. 6A-6B show that Gsx1 induces local network restoration and promotes functional recovery in chronic SCI. Virally transduced cells (GFP) and serotonergic neuronal activity (5-HT) were analyzed in longitudinal spinal cord sections at 56 dpi. FIG. 6A shows Relative Intensity of 5-HT+ cells through the lesion epicenter. FIG. 6B shows impact of AAV6- and LV-mediated Gsx1 treatment on functional recovery after chronic contusion SCI. Data are expressed as mean±SEM. *p<0.05, **p<0.01, AAV-Gsx1 and LV-Gsx1-GFP versus the control group. Statistical analysis was performed using a two-way repeated measures ANOVA followed by Tukey's post hoc test.

FIGS. 7A-7D illustrate laminectomy, spinal cord injury, and gene therapy administration methods. FIG. 7A is a schematic of an exemplary timeline of Gsx1 efficacy experiments including three timepoints for analysis: 4 dpi, 14 dpi, and 56 dpi. Behavioral scoring and urinalysis were conducted throughout the entire 56-day study. FIG. 7B is an outline of experimental stages for Gsx1 treatment and histological/behavioral analyses. FIG. 7C is a schematic representation of spinal cord injury and gene therapy administration in both the lateral hemisection and contusion injury models. FIG. 7D shows injection depths for gene delivery in the injured rat spinal cord.

FIGS. 8A-8I show quantification of Gsx1 induced activation, proliferation, and neurogenesis in rats with lateral hemisection SCI at 4 dpi. The number of co-labeled cells adjacent to the injury and injection site are shown for Nestin+GFP+co-labeled cells (FIG. 8A), PCNA+GFP+co-labeled cells (FIG. 8B), and Nestin+PCNA+GFP+co-labeled cells (FIG. 8C). The percentage of co-labeled cells over DAPI+ cells adjacent to the injury and injection site are shown for Nestin+GFP+co-labeled cells (FIG. 8D), PCNA+GFP+co-labeled cells (FIG. 8E), and Nestin+PCNA+GFP+co-labeled cells (FIG. 8F). The percentage of co-labeled cells over GFP+ cells adjacent to the injury and injection site are shown for Nestin+GFP+co-labeled cells (FIG. 8G), PCNA+GFP+co-labeled cells (FIG. 8H), and Nestin+PCNA+GFP+co-labeled cells (FIG. 8I). Data are expressed as mean±SEM. *p<0.05, **p<0.01, AAV6-Gsx1 and LV-Gsx1-GFP versus the control groups (AAV6-GFP, LV-GFP). Statistical analysis was performed using a one-way ANOVA followed by Tukey's post hoc test.

FIGS. 9A-9I show quantification of Gsx1 induced activation, proliferation, and neurogenesis in rats with contusion SCI at 4 dpi. The number of co-labeled cells in an area of 436 μm×328 μm region at/near injury and injection site are shown for Nestin+GFP+co-labeled cells (FIG. 9A), PCNA+GFP+co-labeled cells (FIG. 9B), and Nestin+PCNA+GFP+co-labeled cells (FIG. 9C). The percentage of co-labeled cells over DAPI+ cells in an area of 436 μm×328 μm region at/near injury and injection site are shown for Nestin+GFP+co-labeled cells (FIG. 9D), PCNA+GFP+co-labeled cells (FIG. 9E), and Nestin+PCNA+GFP+co-labeled cells (FIG. 9F). The percentage of co-labeled cells over GFP+ cells in an area of 436 μm×328 μm region at/near injury and injection site are shown for Nestin+GFP+co-labeled cells (FIG. 9G), PCNA+GFP+co-labeled cells (FIG. 9H), and Nestin+PCNA+GFP+co-labeled cells (FIG. 9I). Data are expressed as mean±SEM. *p<0.05, **p<0.01, AAV6-Gsx1 and LV-Gsx1-GFP versus the control group (AAV6-GFP). Statistical analysis was performed using a one-way ANOVA followed by Tukey's post hoc test.

FIGS. 10A-10B show AAV6-Gsx1 activates NG2 progenitors in rats with contusion SCI at 4 dpi. Virally transduced cells (GFP) and NG2 progenitors (NG2) were analyzed in longitudinal spinal cord sections around T9-T10 at 4 dpi. FIG. 10A shows percentage of NG2+ cells over total cells adjacent to the lesion epicenter. FIG. 10B shows percentage of NG2+GFP+ cells over total GFP+ cells adjacent to the lesion epicenter. Data were expressed as mean±SEM. *p<0.05, **p<0.01, AAV6-Gsx1 and LV-Gsx1-GFP versus the control group (AAV6-GFP). Statistical analysis was performed using a one-way ANOVA followed by Tukey's post hoc test.

FIGS. 11A-11B show AAV6-Gsx1 activates Sox2 progenitors in rats with contusion SCI at 4 dpi. Virally transduced cells (GFP) and NSPCs (Nestin) were analyzed in longitudinal spinal cord sections around T9-T10 at 4 dpi. FIG. 11A shows percentage of Sox2+ cells over total cells and FIG. 11B shows Sox2+GFP+ cells over total GFP+ cells adjacent to the lesion epicenter. Data are expressed as mean±SEM. *p<0.05, **p<0.01, AAV6-Gsx1 and LV-Gsx1-GFP versus the control group (AAV6-GFP). Statistical analysis was performed using a one-way ANOVA followed by Tukey's post hoc test.

FIGS. 12A-12B show comparison of viral transduction efficiency in acute and subacute contusion SCI. Quantification of GFP+ virally transduced cells adjacent to the lesion epicenter was performed using Ilastik machine learning-based pixel classification analysis: GFP+ cells over total cells at 4 dpi (FIG. 12A) and 14 dpi (FIG. 12B). Data are expressed as mean±SEM. *p<0.05, **p<0.01, AAV6-Gsx1 and LV-Gsx1-GFP versus the control group (AAV6-GFP). Statistical analysis was performed using a one-way ANOVA followed by Tukey's post hoc test.

FIGS. 13A-13B show analysis of molecular markers in acute contusion SCI. Quantification of molecular markers adjacent to the lesion epicenter was performed using Ilastik machine learning-based pixel classification analysis for Nestin+ cells (FIG. 13A) and PCNA+ cells (FIG. 13B) over total cells at 4 dpi. Data are expressed as mean±SEM. *p<0.05, **p<0.01, AAV6-Gsx1 and LV-Gsx1-GFP versus the control group (AAV6-GFP). Statistical analysis was performed using a one-way ANOVA followed by Tukey's post hoc test.

FIGS. 14A-14C show Gsx1 expression promotes locomotor functional recovery in rats with contusion SCI. Plot of Basso Beattie and Bresnahan (BBB) scores over 56 dpi beginning one day before injury (FIG. 14A) and with individual animals (FIGS. 14B-14C). Data are expressed as mean±SEM. *p<0.05, **p<0.01, ***p<0.001, SCI+AAV6-Gsx1 and SCI+LV-Gsx1-GFP versus the control group (SCI+AAV6-GFP, Sham). Statistical analysis was performed using a two-way repeated measures ANOVA followed by Tukey's post hoc test.

FIGS. 15A-15C show AAV6-Gsx1 and LV-Gsx1 infect mature neurons but do not enhance neuronal survival in rats with contusion SCI at 14 dpi. Virally transduced cells (GFP) and mature neurons (NeuN) were analyzed in longitudinal spinal cord sections around T9-T10 at 14 dpi and percentage of NeuN+ cells over total cells 2 mm away from the lesion epicenter is shown in FIG. 15A. For FIGS. 15B and 15C, virally transduced cells (GFP), mature neurons (MAP2), and cell death (Caspase-3) were analyzed in longitudinal spinal cord sections around T9-T10 at 14 dpi. FIG. 15B shows percentage of GFP+MAP2+Caspase-3+ cells over total GFP+ cells 2 mm away from the lesion epicenter. FIG. 15C shows percentage of GFP+MAP2+ cells over total GFP+ cells 2 mm away from the lesion epicenter. Data were expressed as mean±SEM. *p<0.05, **p<0.01, AAV6-Gsx1 and LV-Gsx1-GFP versus the control group (AAV6-GFP). Statistical analysis was performed using a one-way ANOVA followed by Tukey's post hoc test.

FIGS. 16A-16C demonstrate that Gsx1 does not change mature neuron myelination, neuronal activity, or synapse formation in rats with contusion SCI at 14 dpi. Virally transduced cells (GFP), myelin (Myelin Basic Protein) and mature neurons (MAP2) were analyzed in longitudinal spinal cord sections around T9-T10 at 14 dpi and FIG. 16A shows percentage of GFP+MAP2+MyelinBasicProtein+ cells over total GFP+ cells 2 mm away from the lesion epicenter. Virally transduced cells (GFP), serotonergic neuronal activity (5-HT) and mature neurons (MAP2) were analyzed in longitudinal spinal cord sections around T9-T10 at 14 dpi and FIG. 16B shows percentage of GFP+MAP2+5-HT+ cells over total GFP+ cells 2 mm away from the lesion epicenter. Virally transduced cells (GFP), synapses (Synaptophysin) and mature neurons (MAP2) were analyzed in longitudinal spinal cord sections around T9-T10 at 14 dpi and FIG. 16C shows percentage of GFP+MAP2+Synaptophysin+ cells over total GFP+ cells 2 mm away from the lesion epicenter. Data were expressed as mean±SEM. *p<0.05, **p<0.01, AAV6-Gsx1 and LV-Gsx1-GFP versus the control group (AAV6-GFP). Statistical analysis was performed using a one-way ANOVA followed by Tukey's post hoc test.

FIG. 17 is a schematic diagram of an exemplary AAV6 vector encoding mouse Gsx1 and green fluorescent protein.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand.

SEQ ID NO: 1 is an exemplary mouse Gsx1 nucleic acid:
CAGCAGCAGCCAAGGTGATTCCAGCCCGGGCTTGAGCCGCGAGTGGAGCCT
CCGGGGCCCGGGAAGCTGCGGGTGGCCGCGGCCAGGGGAAGCTACGACAG
GATCTGCAGTTCCCTCGGGCTCCAGGGGCGGGCTGGCGGCAGGTGGACCTC
GCGCTGGAGCCATGCCGCGCTCCTTCCTGGTGGATTCCCTTGTGCTGCGGGA
AGCCAGCGACAAGAAGGCTCCGGAGGGCAGCCCGCCACCGCTCTTCCCCTA
CGCGGTCCCGCCGCCGCACGCGCTCCACGGCCTCTCGCCGGGCGCCTGCCA
CGCGCGCAAGGCCGGCTTGCTGTGCGTGTGTCCCCTCTGTGTCACCGCTTCG
CAGCTGCACGGGCCCCCCGGGCCGCCGGCACTGCCGCTACTCAAGGCGTCC
TTCCCTCCCTTCGGATCGCAGTACTGCCACGCACCCCTGGGCCGCCAGCACT
CCGTGTCCCCTGGAGTCGCCCACGGCCCGGCCGCGGCCGCAGCAGCAGCTG
CACTCTACCAGACCTCCTACCCGCTGCCGGATCCCAGACAGTTTCACTGCAT
CTCTGTGGACAGCAGCTCGAACCAGCTGCCCAGCAGCAAGAGGATGCGGA
CGGCGTTCACCAGCACACAGCTCCTGGAGCTGGAGCGAGAGTTCGCCTCCA
ACATGTACCTCTCCCGCCTGCGGCGCATCGAGATCGCGACCTATCTGAACC
TGTCCGAGAAGCAGGTGAAGATCTGGTTTCAGAACCGCCGGGTGAAGCAC
AAGAAAGAAGGCAAAGGCAGTAACCACCGCGGCGGAGCTGGGGCGGGGG
CCGGCGGGGGCGCACCGCAAGGCTGCAAGTGCTCTTCGCTCTCCTCAGCCA
AATGCTCAGAGGACGACGACGAATTGCCCATGTCTCCATCTTCCTCCGGGA
AGGATGACAGAGATCTCACAGTCACTCCGTAGGTGCGCCTTTTAGAGGACC
ATTGGTTTCCCCACCCCCCACCCCGACCCTTCCCGCACTTCAAAGACTGGTC
CCCAGGCACCCGCTGGCCAACCGACGGATTTCGTTGGGCTTTGCGGTGGTG
CGCAGCTCTAGGCAAAGCTAAGACCTTAGCAGACACTTGAAGACAGTGCCC
CTGTCCCTTGGGCTTCAGGGTGTTTAGGAGGACTCCAAGCGATGAAGGCTG
AGTCCTCCTCCTAGGACACAGCCTCTTCTCCCAGGCACGCAGGCCGGAGCA
CAGCGCCTTGCTGACCGCCAGCGCCTCTTCGCCTGCCAACTCTGGGCTGGTT
CAAGCTTCCTCGGTTCCACTAGTCTCTCCTTCTCGGTCAATCTGGGCTTTTCA
CTCCGCGAGTGGCTGTTTGCTTTTCTTTTAACATTTCTTTCTTGCCCCCAACT
CGTCTCCCCCCACTGTGGTCCTTTATGCAACACGTCTATGGACTTAACTTTT
CCTTCCCTCCTCAGGAAGTCTCTCCCTTCTGTCCGTTTGTCCC
SEQ ID NO: 2 is an exemplary mouse Gsx1 protein coding sequence:
ATGCCGCGCTCCTTCCTGGTGGATTCCCTTGTGCTGCGGGAAGCCAGCGACA
AGAAGGCTCCGGAGGGCAGCCCGCCACCGCTCTTCCCCTACGCGGTCCCGC
CGCCGCACGCGCTCCACGGCCTCTCGCCGGGCGCCTGCCACGCGCGCAAGG
CCGGCTTGCTGTGCGTGTGTCCCCTCTGTGTCACCGCTTCGCAGCTGCACGG
GCCCCCCGGGCCGCCGGCACTGCCGCTACTCAAGGCGTCCTTCCCTCCCTTC
GGATCGCAGTACTGCCACGCACCCCTGGGCCGCCAGCACTCCGTGTCCCCTG
GAGTCGCCCACGGCCCGGCCGCGGCCGCAGCAGCAGCTGCACTCTACCAGA
CCTCCTACCCGCTGCCGGATCCCAGACAGTTTCACTGCATCTCTGTGGACAG
CAGCTCGAACCAGCTGCCCAGCAGCAAGAGGATGCGGACGGCGTTCACCAG
CACACAGCTCCTGGAGCTGGAGCGAGAGTTCGCCTCCAACATGTACCTCTCC
CGCCTGCGGCGCATCGAGATCGCGACCTATCTGAACCTGTCCGAGAAGCAG
GTGAAGATCTGGTTTCAGAACCGCCGGGTGAAGCACAAGAAAGAAGGCAA
AGGCAGTAACCACCGCGGCGGAGCTGGGGCGGGGGCCGGCGGGGGCGCAC
CGCAAGGCTGCAAGTGCTCTTCGCTCTCCTCAGCCAAATGCTCAGAGGACG
ACGACGAATTGCCCATGTCTCCATCTTCCTCCGGGAAGGATGACAGAGATCT
CACAGTCACTCCGTAG
SEQ ID NO: 3 is an exemplary mouse Gsx1 amino acid sequence:
MPRSFLVDSLVLREASDKKAPEGSPPPLFPYAVPPPHALHGLSPGACHARKAGL
LCVCPLCVTASQLHGPPGPPALPLLKASFPPFGSQYCHAPLGRQHSVSPGVAHG
PAAAAAAAALYQTSYPLPDPRQFHCISVDSSSNQLPSSKRMRTAFTSTQLLELER
EFASNMYLSRLRRIEIATYLNLSEKQVKIWFQNRRVKHKKEGKGSNHRGGAGA
GAGGGAPQGCKCSSLSSAKCSEDDDELPMSPSSSGKDDRDLTVTP
SEQ ID NO: 4 is an exemplary human Gsx1 nucleic acid:
ACCACTAGCGCTGGCCAGCACCCCGCGCTCTTTGGGCGGTGCCCACGGCAG
CAGAGGCTACTGTTTCAGCCTAGGTCTCAGCCGCGCGTTCAGCCTCCTGGGC
AGAGGCAGCTGCGGGATACCGCGGCCAGGGAAAGCGCGTGGAGAGCCGAA
AGGTGCGGTGGGCGCAGAGGGCGGGCTGGCTGCGGGGCGACCGCGCGCCG
GGGCCATGCCGCGCTCCTTCCTGGTGGACTCGCTAGTGCTGCGCGAGGCGG
GCGAGAAGAAGGCGCCCGAGGGCAGCCCGCCGCCGCTCTTCCCCTACGCTG
TGCCCCCGCCGCACGCGCTGCACGGTCTCTCGCCTGGCGCCTGCCACGCGCG
CAAGGCTGGGCTGCTGTGCGTGTGCCCGCTCTGCGTCACCGCCTCGCAGCTG
CATGGGCCCCCCGGGCCGCCCGCGCTGCCTCTACTCAAGGCTTCCTTCCCAC
CCTTCGGCTCGCAGTACTGCCACGCGCCCCTGGGCCGCCAGCACTCTGCTGT
GTCGCCCGGGGTCGCTCACGGCCCGGCCGCCGCTGCTGCTGCCGCCGCGCTC
TACCAGACCTCCTACCCGCTGCCTGACCCCAGGCAGTTCCACTGCATCTCTG
TGGACAGCAGCTCTAACCAGCTGCCCAGCAGCAAGAGGATGCGCACGGCTT
TCACCAGCACGCAGCTGCTAGAGCTGGAGCGCGAGTTCGCTTCTAATATGT
ACCTGTCCCGCCTACGTCGCATCGAGATCGCGACCTACCTGAATCTGTCCGA
GAAGCAGGTGAAGATCTGGTTTCAGAACCGCCGAGTGAAGCACAAGAAGG
AGGGCAAGGGCAGCAACCATCGTGGCGGCGGCGGCGGGGGTGCCGGTGGT
GGCGGGAGCGCACCGCAAGGCTGCAAGTGCGCATCGCTCTCCTCAGCCAAG
TGCTCCGAGGATGACGACGAATTGCCCATGTCTCCGTCCTCCTCAGGGAAG
GACGACCGGGATCTTACGGTCACTCCCTAGGCGCGTGTCTCCCTAGGTCGCC
CACCCCAAGACCTCCCTGCGCCTCGGAGACTAGTCCTGGGACTCAGCGCTG
ATTCCCAGGCACCCGCAGCCAAACCACTGCCTGGCATGGATTTGGCACTGCT
TTGCAGAGGTCCCGGGCCTGGGCAGGGCTGAGAGCTTGGCAGAGACTGGAC
CATGGCGTCCCCGCCCCAGGGCTCGCTCGTGTCCAAAGCCAACTCCAAGCT
GTGAACACTGTAAGCGCTCGAGTCCTCCTGGGCAGTGCAGCACCGCGGCCC
CCGCCCGCAAGCCTGCTTGGCGCAGCGCCTTGCTGGCAGCCGGCGCCTCCTA
GCCCGCCCTCTCTGGGCTGGCTTGGGCTTCCTCGCTTACTCTACTCTCCTCCG
CTCTCGGTCAAGGTCGGCGGCCTTGGATGCTCGTTCGCTTCTCTTTTTAAAAT
GTCTTTTTGTCCCTCCCCACCTCTTTCCTGTGATCATTTATTCACTCGGCCCC
CGCCCGCCAACACACACATTTATGAACCCCCACTTTTTTTTTCCTTCTCCTTT
TCCCTTCCCCTCCTCAGGGAGCCTCCTCCTTCAGTCAGTCCATTTGTCCCTTG
ATCTGGCCTTGCTGTCCTCAGTCCCCACGGCTCCTCTCACAGATGATAAATT
TCGCCCGTAGTATCCATATTGGGGAAACAGATTTGCTCTGTTTCCAACACGC
TCTTCCTTCCTCTAAACATAGGACCCCGCTCTGCGCCGCTGGCGACCCCACT
CTTCCCCCTCCAGACTCTGGCCCGCCCCAGCCTAGCTGTGTAATTGTACGGC
CTCTGCAATGCCAGAAG
SEQ ID NO: 5 is an exemplary human Gsx1 protein coding sequence:
ATGCCGCGCTCCTTCCTGGTGGACTCGCTAGTGCTGCGCGAGGCGGGCGAG
AAGAAGGCGCCCGAGGGCAGCCCGCCGCCGCTCTTCCCCTACGCTGTGCCC
CCGCCGCACGCGCTGCACGGTCTCTCGCCTGGCGCCTGCCACGCGCGCAAG
GCTGGGCTGCTGTGCGTGTGCCCGCTCTGCGTCACCGCCTCGCAGCTGCATG
GGCCCCCCGGGCCGCCCGCGCTGCCTCTACTCAAGGCTTCCTTCCCACCCTT
CGGCTCGCAGTACTGCCACGCGCCCCTGGGCCGCCAGCACTCTGCTGTGTCG
CCCGGGGTCGCTCACGGCCCGGCCGCCGCTGCTGCTGCCGCCGCGCTCTACC
AGACCTCCTACCCGCTGCCTGACCCCAGGCAGTTCCACTGCATCTCTGTGGA
CAGCAGCTCTAACCAGCTGCCCAGCAGCAAGAGGATGCGCACGGCTTTCAC
CAGCACGCAGCTGCTAGAGCTGGAGCGCGAGTTCGCTTCTAATATGTACCT
GTCCCGCCTACGTCGCATCGAGATCGCGACCTACCTGAATCTGTCCGAGAA
GCAGGTGAAGATCTGGTTTCAGAACCGCCGAGTGAAGCACAAGAAGGAGG
GCAAGGGCAGCAACCATCGTGGCGGCGGCGGCGGGGGTGCCGGTGGTGGC
GGGAGCGCACCGCAAGGCTGCAAGTGCGCATCGCTCTCCTCAGCCAAGTGC
TCCGAGGATGACGACGAATTGCCCATGTCTCCGTCCTCCTCAGGGAAGGAC
GACCGGGATCTTACGGTCACTCCCTAG
SEQ ID NO: 6 is an exemplary human Gsx1 amino acid sequence:
MPRSFLVDSLVLREAGEKKAPEGSPPPLFPYAVPPPHALHGLSPGACHARKAGL
LCVCPLCVTASQLHGPPGPPALPLLKASFPPFGSQYCHAPLGRQHSAVSPGVAH
GPAAAAAAAALYQTSYPLPDPRQFHCISVDSSSNQLPSSKRMRTAFTSTQLLEL
EREFASNMYLSRLRRIEIATYLNLSEKQVKIWFQNRRVKHKKEGKGSNHRGGG
GGGAGGGGSAPQGCKCASLSSAKCSEDDDELPMSPSSSGKDDRDLTVTP
SEQ ID NO: 7 is an exemplary AAV6-mGsx1-GFP vector:
CCTGCAGGCAGCTGCGCGCTCGCTCGCTCACTGAGGCCGCCCGGGCAAAGC
CCGGGCGTCGGGCGACCTTTGGTCGCCCGGCCTCAGTGAGCGAGCGAGCGC
GCAGAGAGGGAGTGGCCAACTCCATCACTAGGGGTTCCTGCGGCCTAAGGC
AATTGCGATGTACGGGCCAGATATACGCGTTGACATTGATTATTGACTAGTT
ATTAATAGTAATCAATTACGGGGTCATTAGTTCATAGCCCATATATGGAGTT
CCGCGTTACATAACTTACGGTAAATGGCCCGCCTGGCTGACCGCCCAACGA
CCCCCGCCCATTGACGTCAATAATGACGTATGTTCCCATAGTAACGCCAATA
GGGACTTTCCATTGACGTCAATGGGTGGAGTATTTACGGTAAACTGCCCACT
TGGCAGTACATCAAGTGTATCATATGCCAAGTACGCCCCCTATTGACGTCAA
TGACGGTAAATGGCCCGCCTGGCATTATGCCCAGTACATGACCTTATGGGA
CTTTCCTACTTGGCAGTACATCTACGTATTAGTCATCGCTATTACCATGGTG
ATGCGGTTTTGGCAGTACATCAATGGGCGTGGATAGCGGTTTGACTCACGG
GGATTTCCAAGTCTCCACCCCATTGACGTCAATGGGAGTTTGTTTTGGCACC
AAAATCAACGGGACTTTCCAAAATGTCGTAACAACTCCGCCCCATTGACGC
AAATGGGCGGTAGGCGTGTACGGTGGGAGGTCTATATAAGCAGAGCTCTCT
GGCTAACTAGAGAACCCACTGCTTACTGGCTTATCGAAATTGTCGACAAGCT
AGCCAAAGCTTCAGGTACCAGGGATCCACGAATTCGCCCTTcagcagcagccaaggt
gattccagcccgggcttgagccgcgagtggagcctccggggcccgggaagctgcgggtggccgcggccaggggaagct
acgacaggatctgcagttccctcgggctccaggggcgggctggcggcaggtggacctcgcgctggagccatgccgcgctc
cttcctggtggattcccttgtgctgcgggaagccagcgacaagaaggctccggagggcagcccgccaccgctcttcccctac
gcggtcccgccgccgcacgcgctccacggcctctcgccgggcgcctgccacgcgcgcaaggccggcttgctgtgcgtgtg
tcccctctgtgtcaccgcttcgcagctgcacgggccccccgggccgccggcactgccgctactcaaggcgtccttccctccct
tcggatcgcagtactgccacgcacccctgggccgccagcactccgtgtcccctggagtcgcccacggcccggccgcggcc
gcagcagcagctgcactctaccagacctcctacccgctgccggatcccagacagtttcactgcatctctgtggacagcagctc
gaaccagctgcccagcagcaagaggatgcggacggcgttcaccagcacacagctcctggagctggagcgagagttcgcct
ccaacatgtacctctcccgcctgcggcgcatcgagatcgcgacctatctgaacctgtccgagaagcaggtgaagatctggttt
cagaaccgccgggtgaagcacaagaaagaaggcaaaggcagtaaccaccgcggcggagctgggggggggccggcg
ggggcgcaccgcaaggctgcaagtgctcttcgctctcctcagccaaatgctcagaggacgacgacgaattgcccatgtctcc
atcttcctccgggaaggatgacagagatctcacagtcactccgtaggtgcgccttttagaggaccattggtttccccacccccc
accccgacccttcccgcacttcaaagactggtccccaggcacccgctggccaaccgacggatttcgttgggctttgcggtggt
gcgcagctctaggcaaagctaagaccttagcagacacttgaagacagtgcccctgtcccttgggcttcagggtgtttaggagg
actccaagcgatgaaggctgagtcctcctcctaggacacagcctcttctcccaggcacgcaggccggagcacagcgccttg
ctgaccgccagcgcctcttcgcctgccaactctgggctggttcaagcttcctcggttccactagtctctccttctcggtcaatctg
ggcttttcactccgcgagtggctgtttgcttttttttaacatttctttcttgcccccaactcgtctccccccactgtggtcctttatgca
acacgtctatggacttaacttttccttccctcctcaggaagtctctcccttctgtccgtttgtcccGTCGCTCCAAGGG
CGAATTCACGATATCAAGCGGCCGCAACTCGAGACTCTAGACGACTGTGCC
TTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGG
TGGGGAATGTGTGTCAGTTAGGGTGTGGAAAGTCCCCAGGCTCCCCAGCAG
GCAGAAGTATGCAAAGCATGCATCTCAATTAGTCAGCAACCAGGTGTGGAA
AGTCCCCAGGCTCCCCAGCAGGCAGAAGTATGCAAAGCATGCATCTCAATT
AGTCAGCAACCATAGTCCCGCCCCTAACTCCGCCCATCCCGCCCCTAACTCC
GCCCAGTTCCGCCCATTCTCCGCCCCATGGCTGACTAATTTTTTTTATTTATG
CAGAGGCCGAGGCCGCCTCTGCCTCTGAGCTATTCCAGAAGTAGTGAGGAG
GCTTTTTTGGAGGCCTAGGCTTTTGCAAAAAGCTCCGCGGAGATCTAAGCTC
TAGCCACCATGGTGAGCAAGGGCGAGGAGCTGTTCACCGGGGTGGTGCCCA
TCCTGGTCGAGCTGGACGGCGACGTAAACGGCCACAAGTTCAGCGTGTCCG
GCGAGGGCGAGGGCGATGCCACCTACGGCAAGCTGACCCTGAAGTTCATCT
GCACCACCGGCAAGCTGCCCGTGCCCTGGCCCACCCTCGTGACCACCCTGA
CCTACGGCGTGCAGTGCTTCAGCCGCTACCCCGACCACATGAAGCAGCACG
ACTTCTTCAAGTCCGCCATGCCCGAAGGCTACGTCCAGGAGCGCACCATCTT
CTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGA
CGGCAACATCCTGGGGCACAAGCTGGAGTACAACTACAACAGCCACAACGT
CTATATCATGGCCGACAAGCAGAAGAACGGCATCAAGGTGAACTTCAAGAT
CCGCCACAACATCGAGGACGGCAGCGTGCAGCTCGCCGACCACTACCAGCA
GAACACCCCCATCGGCGACGGCCCCGTGCTGCTGCCCGACAACCACTACCT
GAGCACCCAGTCCGCCCTGAGCAAAGACCCCAACGAGAAGCGCGATCACAT
GGTCCTGCTGGAGTTCGTGACCGCCGCCGGGATCACTCTCGGCATGGACGA
GCTGTACAAGTAAGTTTAAACCTCGATCGAGTCTAGATCAATATTTCGAGTA
CCTCTAGGGCCGCTTCGAGCAGACATGATAAGATACATTGATGAGTTTGGA
CAAACCACAACTAGAATGCAGTGAAAAAAATGCTTTATTTGTGAAATTTGT
GATGCTATTGCTTTATTTGTAACCATTATAAGCTGCAATAAACAAGTTAACA
ACAACAATTGCATTCATTTTATGTTTCAGGTTCAGGGGGAGATGTGGGAGGT
TTTTTAAAGCAAGTAAAACCTCTACAAATGTGGTAAAATCGAATCTAGATCC
TCTCTTAAGGGCCGCAGGAACCCCTAGTGATGGAGTTGGCCACTCCCTCTCT
GCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGCCTGCA
GGGGCGCCTGATGCGGTATTTTCTCCTTACGCATCTGTGCGGTATTTCACAC
CGCATACGTCAAAGCAACCATAGTACGCGCCCTGTAGCGGCGCATTAAGCG
CGGCGGGTGTGGTGGTTACGCGCAGCGTGACCGCTACACTTGCCAGCGCCC
TAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTG
CTTTACGGCACCTCGACCCCAAAAAACTTGATTTGGGTGATGGTTCACGTAG
TGGGCCATCGCCCTGATAGACGGTTTTTCGCCCTTTGACGTTGGAGTCCACG
TTCTTTAATAGTGGACTCTTGTTCCAAACTGGAACAACACTCAACCCTATCT
CGGGCTATTCTTTTGATTTATAAGGGATTTTGCCGATTTCGGCCTATTGGTTA
AAAAATGAGCTGATTTAACAAAAATTTAACGCGAATTTTAACAAAATATTA
ACGTTTACAATTTTATGGTGCACTCTCAGTACAATCTGCTCTGATGCCGCAT
AGTTAAGCCAGCCCCGACACCCGCCAACACCCGCTGACGCGCCCTGACGGG
CTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAA
GGGCCTCGTGATACGCCTATTTTTATAGGTTAATGTCATGATAATAATGGTT
TCTTAGACGTCAGGTGGCACTTTTCGGGGAAATGTGCGCGGAACCCCTATTT
GTTTATTTTTCTAAATACATTCAAATATGTATCCGCTCATGAGACAATAACC
CTGATAAATGCTTCAATAATATTGAAAAAGGAAGAGTATGAGTATTCAACA
TTTCCGTGTCGCCCTTATTCCCTTTTTTGCGGCATTTTGCCTTCCTGTTTTTGC
TCACCCAGAAACGCTGGTGAAAGTAAAAGATGCTGAAGATCAGTTGGGTGC
ACGAGTGGGTTACATCGAACTGGATCTCAACAGCGGTAAGATCCTTGAGAG
TTTTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCGC
CGCATACACTATTCTCAGAATGACTTGGTTGAGTACTCACCAGTCACAGAAA
AGCATCTTACGGATGGCATGACAGTAAGAGAATTATGCAGTGCTGCCATAA
CCATGAGTGATAACACTGCGGCCAACTTACTTCTGACAACGATCGGAGGAC
CGAAGGAGCTAACCGCTTTTTTGCACAACATGGGGGATCATGTAACTCGCCT
TGATCGTTGGGAACCGGAGCTGAATGAAGCCATACCAAACGACGAGCGTGA
CACCACGATGCCTGTAGCAATGGCAACAACGTTGCGCAAACTATTAACTGG
CGAACTACTTACTCTAGCTTCCCGGCAACAATTAATAGACTGGATGGAGGC
GGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCA
GCACTGGGGCCAGATGGTAAGCCCTCCCGTATCGTAGTTATCTACACGACG
GGGAGTCAGGCAACTATGGATGAACGAAATAGACAGATCGCTGAGATAGG
TGCCTCACTGATTAAGCATTGGTAACTGTCAGACCAAGTTTACTCATATATA
CTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCAC
TGAGCGTCAGACCCCGTAGAAAAGATCAAAGGATCTTCTTGAGATCCTTTTT
TTCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACTGG
CTTCAGCAGAGCGCAGATACCAAATACTGTCCTTCTAGTGTAGCCGTAGTTA
GGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGG
GGGGTTCGTGCACACAGCCCAGCTTGGAGCGAACGACCTACACCGAACTGA
GATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACG
AGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGC
TGGCCTTTTGCTCACATGT
SEQ ID NO: 8 is an exemplary rat Gsx1 nucleic acid sequence:
CCAGCACCTCGCGCGCTCCGGGCGGCGCCCGCAGCAGCAGCCAAGGTGATT
CCAGCCCCGGCTTGAGCCGCGCGTGGAGCCTCCCGGACCCGGGAAACTGCG
GGTGGCCGCGGCAGAGGGAGGCCACTGCAGAATCCGCAGTTCCCTCGGGCG
CCAGGGGCGGGCTGGCAGCAGGTGGACCGCGCGCCGGAGCCATGCCGCGCT
CTTTCCTGGTGGATTCCCTTGTGCTGCGGGAAGCCAGCGACAAGAAGGCTCC
GGAGGGCAGCCCGCCACCGCTCTTCCCCTACGCGGTCCCCCCGCCGCACGC
GCTCCACGGCCTCTCGCCGGGCGCCTGCCACGCGCGCAAGGCCGGCTTGCT
GTGCGTGTGTCCCCTCTGTGTCACCGCTTCGCAGCTGCACGGGCCCCCCGGG
CCGCCGGCGCTGCCGCTACTCAAGGCGTCCTTCCCTCCCTTCGGATCGCAGT
ACTGCCACGCACCCCTGGGCCGCCAGCACTCTGTGTCTCCCGGAGTCGCCCA
CAGCCCGGCCGCGGCTGCAGCAGCTGCCGCACTCTACCAGACCTCCTACCC
GCTGCCGGATCCCAGACAGTTTCACTGCATCTCCGTGGACAGCAGCTCGAA
CCAGCTGCCCAGCAGTAAGAGGATGCGGACGGCGTTCACCAGCACGCAGCT
CCTGGAGCTGGAGCGCGAGTTCGCCTCCAACATGTACCTCTCCCGCCTGCGG
CGCATCGAGATCGCGACCTATCTGAATCTGTCCGAGAAGCAGGTGAAGATC
TGGTTTCAGAACCGCCGGGTGAAGCACAAGAAAGAAGGCAAGGGCAGTAA
CCACCGCGGCGGAGCTGGGGCGGGGGCCGGCGGGGGCGCACCGCAAGGCT
GCAAGTGCTCTTCGCTCTCCTCAGCCAAATGCTCAGAGGATGACGACGAATT
GCCCATGTCTCCATCTTCCTCCGGGAAGGATGACAGAGATCTCACAGTCACT
CCGTAGGTGCGCCTTTTAGAGGACCATTGGTCCCCCCCCTTCCCCGGCCCTT
CCCACACTTCAAAGACTGGTTCTTAGGCTCCGGCTGCCAATCAATGGATGGG
GAGGGCTTTGCGGTGGTGCACAGCTCTAGGCAGAGCTGAGAC
CTTAGCAGAGACTTGAAGCCCTTGTCACTAGGGCTTCAGGGTGTTTAGGAG
GACTCCAAATGGTGAACGCTGGGTCCTCCTCCCAGGACACAGCTTCTTCTCC
CCCCCCAGGCACGCAGGCCGGAGAACAGCGCCTTGCTGACCGCCGGCGCCT
CCTCGCCGCTAACTCTGGACTGGTTCAGGCTTCCTCTATCCCACAACCCTCT
CTTCCTCGGTCAAGCTGGGCTTTTTCACTCCGCTAGTGGCTATTTGCTTTCCT
TTTAACATTTTTTCTTGTTCTCACTCCCCCACAGCCCCCCCCGTGGTCGTTTA
TGCAACGCGTTTGTTACCCCCCCCCCCACACACACACACACTCCGGAAGCCT
CTTCCTTCTGTCTGTTTGTCCCTTAAACAGGGACCAGGTCTTGCTGTTCGAAG
AAGTCCCCTTAGCAGAGAGGACTGTCTCAAATGGTATTGTATTGGGGGAAA
TGACTGTTTCCAACACTCTCCACCCCCTTTCAAATGAAGCCGCTGTAAACAA
CCTCTCCCCCATCGTCCAGGTCCCGACCCCTTCTGGGGACTGACTGACTGTG
TTGTTGTATGGTCTCTGTAATGCCAGAAGATATTTATTTATTTATTTATGTAC
AAAATTTTAAATAAACTTTTTTTTCTTAGAAA
SEQ ID NO: 9 is an exemplary rat Gsx1 protein coding sequence:
ATGCCGCGCTCTTTCCTGGTGGATTCCCTTGTGCTGCGGGAAGCCAGCGACA
AGAAGGCTCCGGAGGGCAGCCCGCCACCGCTCTTCCCCTACGCGGTCCCCC
CGCCGCACGCGCTCCACGGCCTCTCGCCGGGCGCCTGCCACGCGCGCAAGG
CCGGCTTGCTGTGCGTGTGTCCCCTCTGTGTCACCGCTTCGCAGCTGCACGG
GCCCCCCGGGCCGCCGGCGCTGCCGCTACTCAAGGCGTCCTTCCCTCCCTTC
GGATCGCAGTACTGCCACGCACCCCTGGGCCGCCAGCACTCTGTGTCTCCCG
GAGTCGCCCACAGCCCGGCCGCGGCTGCAGCAGCTGCCGCACTCTACCAGA
CCTCCTACCCGCTGCCGGATCCCAGACAGTTTCACTGCATCTCCGTGGACAG
CAGCTCGAACCAGCTGCCCAGCAGTAAGAGGATGCGGACGGCGTTCACCAG
CACGCAGCTCCTGGAGCTGGAGCGCGAGTTCGCCTCCAACATGTACCTCTCC
CGCCTGCGGCGCATCGAGATCGCGACCTATCTGAATCTGTCCGAGAAGCAG
GTGAAGATCTGGTTTCAGAACCGCCGGGTGAAGCACAAGAAAGAAGGCAA
GGGCAGTAACCACCGCGGCGGAGCTGGGGCGGGGGCCGGCGGGGGCGCAC
CGCAAGGCTGCAAGTGCTCTTCGCTCTCCTCAGCCAAATGCTCAGAGGATG
ACGACGAATTGCCCATGTCTCCATCTTCCTCCGGGAAGGATGACAGAGATCT
CACAGTCACTCCGTAG
SEQ ID NO: 10 is an exemplary rat Gsx1 amino acid sequence:
MPRSFLVDSLVLREASDKKAPEGSPPPLFPYAVPPPHALHGLSPGACHARKAGL
LCVCPLCVTASQLHGPPGPPALPLLKASFPPFGSQYCHAPLGRQHSVSPGVAHSP
AAAAAAAALYQTSYPLPDPRQFHCISVDSSSNQLPSSKRMRTAFTSTQLLELER
EFASNMYLSRLRRIEIATYLNLSEKQVKIWFQNRRVKHKKEGKGSNHRGGAGA
GAGGGAPQGCKCSSLSSAKCSEDDDELPMSPSSSGKDDRDLTVTP

### DETAILED DESCRIPTION

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a protein” includes singular or plural proteins and can be considered equivalent to the phrase “at least one protein.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy.

Administration: To provide or give a subject an agent, such as an adeno-associated virus 6 (AAV6) vector comprising a heterologous nucleic acid (such as a heterologous nucleic acid encoding Gsx1), by any effective route. Exemplary routes of administration include, but are not limited to, injection (such as injection into the CNS, for example injection into the spine or brain, for example at or near a site of injury, for example rostral and/or caudal to the injury site). In some examples, administration is an intrathecal injection to treat SCI in lumbar/sacral region, a cisterna magna injection to treat SCI in cervical/thoracic region, or intraparenchymal or introcerebroventricular injection to treat traumatic brain injury.

Contact: Placement in direct physical association, including a solid or a liquid form. Contacting can occur in vitro or ex vivo, for example, by adding a reagent to a sample (such as one containing neural cells), or in vivo by administering to a subject.

Effective amount: The amount of an agent (such as an adeno-associated virus 6 (AAV6) vector comprising a heterologous nucleic acid) that is sufficient to effect beneficial or desired results.

A therapeutically effective amount may vary depending upon one or more of: the subject and disease condition being treated, the weight and age of the subject, the severity of the disease condition, the manner of administration and the like, which can be determined by one of ordinary skill in the art. The beneficial therapeutic effect can include amelioration of a disease, symptom, disorder, or pathological condition; reducing or preventing the onset of a disease, symptom, disorder or condition; and generally counteracting a disease, symptom, disorder or pathological condition.

Expression: The process by which the coded information of a nucleic acid molecule, such as a Gsx1 nucleic acid molecule is converted into an operational, non-operational, or structural part of a cell, such as the synthesis of a protein (e.g., Gsx1 protein). Expression of a gene can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule or protein can be altered relative to a normal (wild type) nucleic acid molecule or protein (such as in a normal non-recombinant cell). Alterations in gene expression, such as differential expression, include but are not limited to: (1) overexpression (e.g., upregulation); (2) underexpression (e.g., downregulation); or (3) suppression of expression. Alternations in the expression of a nucleic acid molecule can be associated with, and in fact cause, a change in expression of the corresponding protein.

GS Homeobox 1 (GSX1): (e.g., OMIM 616542): Also known as Gsh1. The mouse protein is 261 amino acids, and the human protein is 264 amino acids, and the two proteins share about 97% sequence identity. The human GSX1 gene maps to chromosome 13q12.2.

Gsx1 sequences are publicly available, for example from the GenBank® sequence database (e.g., Accession Nos. NP_663632.1, NP_032204.1, XP_006068096.2, NP_001178592.1, and NP_001178592.1 provide exemplary Gsx1 protein sequences, while Accession Nos. NM_145657.3, NM_008178.3, XM_006068034.4, NM_001191663.2, and NM_001191663.2 provide exemplary Gsx1 nucleic acid sequences). One of ordinary skill in the art can identify additional Gsx1 nucleic acid and protein sequences, including Gsx1 variants, such as those having at least 80%, at least 85%, at least 90%, at least 92%, at least 95%, at least 98%, or at least 99% sequence identity to these GenBank® sequences.

Increase or Decrease: A statistically significant positive or negative change, respectively, in quantity from a control value. An increase is a positive change, such as an increase at least 50%, at least 100%, at least 200%, at least 300%, at least 400% or at least 500% as compared to a control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95% or no more than 99%.

Isolated: An “isolated” biological component (such as a protein or nucleic acid, or cell) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins (such as Gsx1 proteins and nucleic acid molecules) prepared by recombinant expression in a host cell as well as chemically synthesized nucleic acids and proteins. Isolated proteins, nucleic acids, or cells in some examples are at least 50% pure, such as at least 75%, at least 80%, at least 90%, at least 95%, at least 98%, or at least 100% pure.

Non-naturally occurring or engineered: Terms used herein as interchangeably and indicate the involvement of human intervention. The terms, when referring to nucleic acid molecules or polypeptides indicate that the nucleic acid molecule or the polypeptide is at least substantially free from at least one other component with which they are naturally associated in nature and as found in nature. In addition, the terms can indicate that the nucleic acid molecules or polypeptides is one having a sequence not found in nature.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence (such as a Gsx1 coding sequence) if the promoter affects the transcription or expression of the coding sequence. Generally, operably linked DNA sequences are contiguous and, where necessary to join two protein-coding regions, in the same reading frame.

Pharmaceutically acceptable carriers: Pharmaceutically acceptable carriers are known to one of ordinary skill in the art. Remington: The Science and Practice of Pharmacy, Adejare (Ed.), Academic Press, London, United Kingdom, 23^{rd} Edition (2021), describes compositions and formulations suitable for pharmaceutical delivery of recombinant nucleic acid molecules.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide and protein: Refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

Promoter: An array of nucleic acid control sequences which direct transcription of a nucleic acid, such as a Gsx1 coding sequence. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In one example the promoter used is native to the nucleic acid molecule it is expressing (endogenous promoter), for example, is endogenous to Gsx1. In one example the promoter used is not native to the nucleic acid molecule it is expressing (exogenous promoter). A “tissue-specific promoter” is a promoter that direct expression of a nucleic acid molecule in particular cells or tissues, such as the central nervous system. Exemplary promoters that can be used to drive expression of Gsx1 include: CMV promoter, SV40 promoter, or beta actin promoter.

Recombinant or host cell: A cell that has been genetically altered, or is capable of being genetically altered by introduction of an exogenous polynucleotide, such as a recombinant plasmid or vector. Typically, a host cell is a cell in which a recombinant nucleic acid molecule can be propagated and/or its DNA expressed. Such cells can be a neural cell. The term also includes any progeny of the subject host cell. It is understood that all progeny may not be identical to the parental cell since there may be mutations that occur during replication. However, such progeny are included when the term “host cell” is used.

Regulatory element: Includes promoters, enhancers, internal ribosomal entry sites (IRES), and other expression control elements (e.g., transcription termination signals, such as polyadenylation signals and poly-U sequences). Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as neural tissues or cells. Regulatory elements may also direct expression in a temporal-dependent manner, such as in a cell-cycle dependent or developmental stage-dependent manner, which may or may not also be tissue or cell-type specific.

In some embodiments, a Gsx1 coding sequence is operably linked to a promoter, such as a constitutive promoter, such as a pol III promoter, pol II promoter, or pol I promoter. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, CAG promoter, UBC promoter, ROSA promoter, and the EF1α promoter. In some embodiments, a Gsx1 coding sequence is operably linked to a tissue-specific promoter, such as a CNS-specific promoter.

Also encompassed by the term “regulatory element” are enhancer elements, such as WPRE; CMV enhancers; the R-U5′ segment in LTR of HTLV-I (Mol. Cell. Biol., Vol. 8 (1): 466-472, 1988); SV40 enhancer; and the intron sequence between exons 2 and 3 of rabbit β-globin (Proc. Natl. Acad. Sci. USA., 78 (3): 1527-31, 1981). In some embodiments, a Gsx1 coding sequence is operably linked to an enhancer, such as a neural-specific enhancer (e.g., Notch1CR2 or Olig2CR5).

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of protein and nucleic acid sequences (including the Gsx1 sequences provided herein) are typically characterized by possession of at least about 80%, at least 90%, at least 95%, at least 96%, at least 97%, at least 98% or at least 99% sequence identity counted over the full length alignment with the amino acid sequence. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: A mammal, such as a human or veterinary subject. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, boar, bull, horse, or cow. In some examples, the subject is a laboratory animal/organism, such as a mouse, rabbit, or rat. In some examples, the subject has a neurological disorder, such as a neurodegenerative disease or has suffered a brain injury or SCI that can be treated using the methods provided herein.

Transduced, Transformed, Transfected: A virus or vector “transduces” a cell when it transfers nucleic acid molecules into a cell. A cell is “transformed” or “transfected” by a nucleic acid transduced into the cell when the nucleic acid becomes stably replicated by the cell, either by incorporation of the nucleic acid into the cellular genome, or by episomal replication.

These terms encompass all techniques by which a nucleic acid molecule can be introduced into such a cell, including transfection with viral vectors, transformation with plasmid vectors, and introduction of naked DNA by electroporation, lipofection, particle gun acceleration and other methods in the art. In some example the method is a chemical method (e.g., calcium-phosphate transfection or polyethyleneimine (PEI) transfection), physical method (e.g., electroporation, microinjection, particle bombardment), fusion (e.g., liposomes), receptor-mediated endocytosis (e.g., DNA-protein complexes, viral envelope/capsid-DNA complexes) and biological infection by viruses such as recombinant viruses (Wolff, J. A., ed, Gene Therapeutics, Birkhauser, Boston, USA, 1994). Methods for the introduction of nucleic acid molecules into cells are known (e.g., see U.S. Pat. No. 6,110,743).

Treating, Treatment, and Therapy: Any success or indicia of success in the attenuation or amelioration of a pathology or condition, including any objective or subjective parameter such as abatement or diminishing of symptoms. The treatment may be assessed by objective or subjective parameters; including the results of a physical examination, other clinical tests, and the like.

Upregulated: When used in reference to the expression of a molecule, such as a gene or a protein (e.g., Gsx1), refers to any process which results in an increase in production of a gene product. A gene product can be RNA (such as mRNA, rRNA, tRNA, and structural RNA) or protein. Therefore, upregulation includes processes that increase transcription of a gene or translation of mRNA and thus increase the presence of proteins or nucleic acids. The disclosed methods, can be used to upregulate Gsx1.

Examples of processes that increase transcription include those that increase transcription initiation rate, those that increase transcription elongation rate, those that increase processivity of transcription and those that decrease transcriptional repression. Gene upregulation can include increasing expression above an existing level. Examples of processes that increase translation include those that increase translational initiation, those that increase translational elongation and those that increase mRNA stability.

Upregulation includes any detectable increase in the production of a gene product. In certain examples, detectable Gsx1 protein or nucleic acid expression in a cell (such as a cell of the CNS) increases by at least 20%, at least 30%, at least 40%, at least 50%, at least 60%, at least 70%, at least 75%, at least 80%, at least 90%, at least 95%, at least 100%, at least 200%, at least 400%, or at least 500% as compared to a control (such an amount of protein or nucleic acid expression detected in a corresponding normal or non-recombinant cell). In one example, a control is a relative amount of expression in a normal cell (e.g., a non-recombinant CNS cell, such as a neural cell).

Under conditions sufficient for: A phrase that is used to describe any environment that permits a desired activity. In one example the desired activity is expression of a Gsx1 nucleic acid molecule to treat a neurological disorder.

### II. Overview

Spinal cord injury (SCI) is a complex tissue injury resulting in degenerating damage to the central nervous system (CNS) and is characterized by a low quality of life. The clinical pathophysiology of SCI is heterogenous and greatly affected by the extent, location, and type of injury. Immediately following initial mechanical damage, a cascade of cellular/molecular effects occurs resulting in localization of inflammatory cells to the injury site, mass cell apoptosis, release of reactive oxygen species, and glutamate-induced excitotoxicity. Demyelination and neuronal degeneration occur in the mechanically damaged and adjacent spared tissue. The resulting microenvironment is unfavorable for cellular growth and isolated by the glial scar border over a period of weeks.

Neural stem/progenitor cells (NSPCs), characterized by multipotency and self-renewal, are highly diverse with various established markers, e.g., Nestin, Sox2, Foxj1, and NG2. These unique cells produce newborn neurons and glia in the neurogenic niches of the developing and adult CNS. In the injured spinal cord, NSPCs become activated and proliferate to contribute glial fated progeny to the glial scar. NSPCs are a major target for regenerative therapy to treat SCI.

The genomic screened homeobox 1 (Gsx1 or Gsh1) is a neurogenic transcription factor known to regulate the formation of dorsal excitatory and inhibitory spinal cord interneurons during embryonic development. In the adult, the role of inhibitory dorsal interneuron population four is to modulate our perception of pain and itch sensation, whereas excitatory dorsal population five modulates our perception of pain, itch, heat, and touch sensation. Interestingly, the mature dorsal populations formed via Gsx1 expression in the embryo do not contribute to circuits involved in motor function.

The lentivirus (LV) gene delivery method is not ideal. As a retrovirus, the LV incorporates its genome into the host DNA, a process prone to random insertional mutations. The adeno-associated virus (AAV) is a clinically safe alternative as its mechanism of action does not require incorporation of its genome into the host DNA, and thus reduces risk of harm to the patient. A cell specific promoter, e.g., GFAP for astrocytes and NG2 for polydendrocytes, or a particular AAV serotype can be used to target various cell populations in the spinal cord. As disclosed herein, AAV serotype 6 (AAV6) is a highly effective gene delivery system to target NSPCs in the injured spinal cord.

A rat SCI model was utilized herein to select for NSPC-specific AAV serotypes and evaluate Gsx1 therapeutic efficacy. Major differences between the mouse model of SCI and human clinical SCI include increased regenerative capacity in mice, cystic cavity formation in humans, and varying inflammatory reactions. However, in both the human and rat SCI pathophysiology, spontaneous regeneration does not occur and fluid filled cystic cavities form. Thus, a rat model of SCI is more representative of clinical human injury and was used for all experiments.

Most clinical SCI cases are traumatic and occur due to sports, vehicular accidents, and falls. Thus, the contusion/compression SCI type is most representative of clinical pathophysiology. As disclosed herein delivery of both of AAV6- and LV-mediated Gsx1 in both rat models of lateral hemisection and clinically relevant contusion SCI were effective. These findings support the utility of the Gsx1 therapeutic in the heterogeneous clinical setting and provide a delivery method to target NSPCs in the CNS for future therapeutic applications. We also compared commonly used SCI models in the field and provide insight into differences between Gsx1 reactivation in distinct acute SCI types. Promising results in both SCI types serves as evidence that the Gsx1 therapeutic can be used to treat heterogeneous clinical SCI, as the rat models of lateral hemisection and contusion SCI are extremely distinct and contusion injuries occur frequently in the clinic.

### III. Adeno-Associated Viral Vectors

Provided here are methods of expressing a heterologous nucleic acid in NSPCs and methods of treating a neurological disorder in a mammalian subject. The methods include transducing NPSCs or administering to a subject an adeno-associated virus 6 (AAV6) vector or virus including the heterologous nucleic acid molecule.

AAV is a small, non-enveloped helper-dependent parvovirus classified in genus Dependoparvovirus of family Parvoviridae. AAV has a linear, single-stranded DNA genome of about 4.7 kb. The genome is flanked by inverted terminal repeats (ITRs) flanking two open reading frames (ORFs), rep and cap. The rep ORF encodes four replication proteins (Rep78, Rep68, Rep52, and Rep4) and the cap ORF encodes three viral capsid proteins (VP1, VP2, and VP3) and an assembly activating protein (AAP). AAV requires a helper virus (such as adenovirus, herpes simplex virus, or other viruses) to complete its life cycle. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell.

AAV possesses several desirable features for a gene therapy vector, including the ability to bind and enter target cells, enter the nucleus, the ability to be expressed in the nucleus for a prolonged period of time, and low toxicity. Because of the advantageous features of AAV, the present disclosure contemplates the use of AAV6 for use in the methods disclosed herein.

The ITRs are the only component required for successful packaging of a heterologous protein in an AAV capsid. Thus, disclosed herein are AAV6 vectors that include a heterologous nucleic acid operably linked to a promoter. In some examples, the AAV6 vector includes 5′ and 3′ ITRs flanking a heterologous nucleic acid (such as a Gsx1 encoding nucleic acid) operably linked to a promoter.

The vector may also include additional elements, such as an enhancer element (e.g., a nucleic acid sequence that increases the rate of transcription by increasing the activity of a promoter) and/or a polyadenylation signal. In particular examples, the enhancer is a cytomegalovirus (CMV) enhancer or a woodchuck post-transcriptional regulatory element (WPRE).

Exemplary promoters include a constitutive promoter (e.g., CMV, beta actin, or a native Gsx1 promoter), or a tissue-specific promoter, such as a central nervous system (CNS)-specific promoter (e.g., a synapsin 1 (Syn1) promoter, glial fibrillary acidic protein (GFAP) promoter, nestin (NES) promoter, myelin-associated oligodendrocyte basic protein (MOBP) promoter, myelin basic protein (MBP) promoter, tyrosine hydroxylase (TH) promoter, a forkhead box A2 (FOXA2) promoter, a platelet-derived growth factor B chain (PDGF-beta) promoter or neuron-specific enolase (NSE) promoter). In other examples, the promoter is a cell-specific promotor, such as NG2 for polydendrocytes or Foxj1 for ependymal cells. In other example, the promoter is a Notch1CR2 promoter, for example for expression of Gsx1 in Notch1 expressing NSPCs.

In additional examples, the polyadenylation signal is a β-globin polyadenylation signal, an SV40 polyadenylation signal, or a bovine growth hormone (bGH) polyadenylation signal. In one example, the polyadenylation signal is a bGH polyadenylation signal. Other elements that optionally can be included in the vector include tags (such as 6×His, HA, or other tags for protein detection).

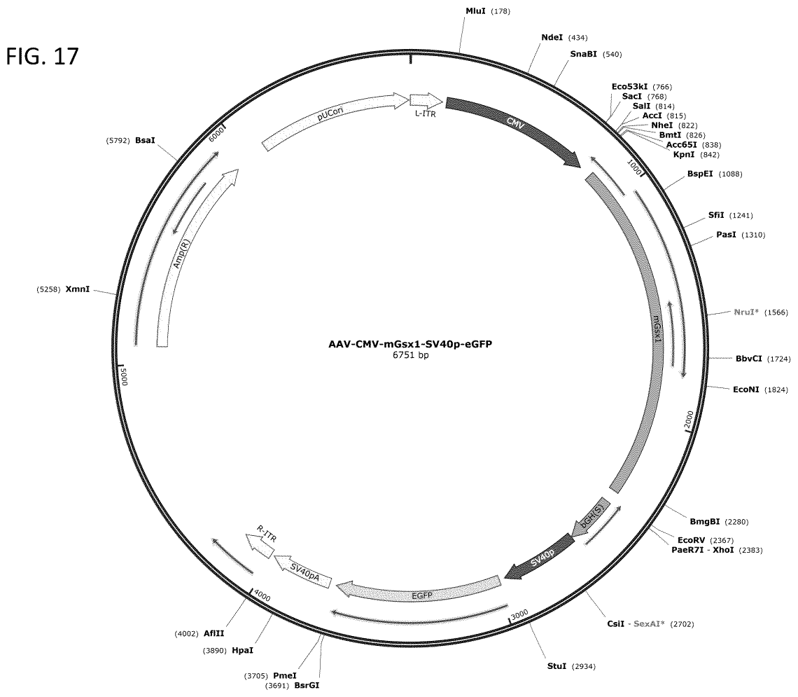

Any combination of ITRs, enhancers, promoters, polyadenylation signals, and/or other elements can be used in the vectors disclosed herein. In some examples, the vector includes (from 5′ to 3′): an AAV6 5′ ITR, a promoter, a heterologous coding sequence, a polyadenylation signal, and an AAV6 3′ ITR. In one non-limiting example, the vector includes (from 5′ to 3′) AAV6 5′ ITR, a CMV promoter, a nucleic acid encoding Gsx1 (for example, SEQ ID NO: 1, SEQ ID NO: 2, SEQ ID NO: 4, SEQ ID NO: 5, SEQ ID NO: 8, or SEQ ID NO: 9), an SV40 polyadenylation signal, and AAV6 3′ ITR (e.g., FIG. 17).

Methods for producing recombinant AAV (rAAV), for example, rAAV suitable for gene therapy are known to one of ordinary skill in the art, and can be utilized with the recombinant nucleic acid molecules, vectors and methods disclosed herein. In some examples, rAAV is produced using a three plasmid system with a plasmid (vector) including the AAV ITRs flanking a promoter operably linked to a nucleic acid encoding a protein of interest (such as a Gsx1-encoding nucleic acid), a plasmid including AAV rep and cap genes operably linked to promoters, and a plasmid encoding helper virus proteins. Cells are cotransfected with the three plasmids and viral assembly occurs. The resulting rAAV particles are purified (for example by gradient centrifugation or HPLC) and can be administered to a subject or are used for transduction of target cells for production of the protein of interest (such as Gsx1). In other examples, a two plasmid system is utilized, with a packaging plasmid (for example including rep and/or cap genes) and a plasmid including the AAV ITRs flanking a promoter operably linked to a nucleic acid encoding a protein of interest (such as a Gsx1-encoding nucleic acid). In this case, additional factors for rAAV production are provided by infection with a helper virus. See, e.g., U.S. Patent Application Publication Nos. 2012/0100606, 2012/0135515, 2011/0229971, and 2013/0225666. In particular examples, the rAAV is serotype AAV6.

Also provided herein are isolated host cells comprising the nucleic acid molecules or vectors disclosed herein. For example, the isolated host cell can be a cell (or cell line) appropriate for production of rAAV. In some examples, the host cell is a mammalian cell, such as a HEK-293 (or HEK293T), BHK, Vero, RD, HT-1080, A549, COS-7, ARPE-19, or MRC-5 cell. One of ordinary skill in the art can select additional cells that can be transformed with the nucleic acids and/or vectors disclosed herein.

### IV. Methods of Treating Neurological Disorders

Provided herein are methods of treating a neurological disorder in a mammalian subject, comprising administering to the subject a therapeutically effective amount of an adeno-associated virus 6 (AAV6) vector or virus comprising a heterologous nucleic acid molecule. In one example, the heterologous nucleic acid is a nucleic acid encoding Gsx1 (for example, SEQ ID NO: 1, SEQ ID NO: 2, SEQ ID NO: 4, SEQ ID NO: 5, SEQ ID NO: 8, or SEQ ID NO: 9).

Exemplary neurological disorders that can be treated with the disclosed methods include spinal cord injuries, brain injuries, or both. In one example, the spinal cord injury, brain injury, or both is caused by trauma from an external force, such as a blow or jolt to the head or a penetrating head injury, such as a vehicle crash (e.g., car, motorcycle, ATV, or bike), fall, act of violence (e.g., gunshot wound or stab wound), or sports (e.g., a collision or fall resulting during football, soccer, baseball, hockey, diving, skiing, rugby, lacrosse, horseback riding, or basketball). A spinal cord injury usually begins with a sudden, traumatic blow to the spine that fractures or dislocates vertebrae. Most injuries to the spine do not completely sever it, but instead cause fracture or compressions of the vertebrae, which then crush and destroy the axons that carry signals up and down the spinal cord. The spinal cord injury can be at the cervical, thoracic, lumbar, sacral, or coccyx region of the spine, such as a C4, C6, T6, T9, T10, or L1 injury. Thus, in some examples, the subject treated with the disclosed methods has quadriplegia or paraplegia.

In one example, the neurological disorder is a traumatic brain injury (TBI), which occurs due to a sudden acceleration or deceleration with the cranium or a combination of movement and sudden impact. Damage occurs both at the time of injury, as well as minutes to days later, for example, due to changes in blood flow and pressure within the cranium. TBI is classified from mild (including concussion) to severe.

In other examples, the neurological disorder that can be treated with the disclosed methods is a neurodegenerative disorder, such as Parkinson's disease, Alzheimer's disease, stroke, ischemia, epilepsy, Huntington's disease, multiple sclerosis, or amyotrophic lateral sclerosis. Such neurodegenerative disorders are an abnormality in the nervous system of a mammalian subject, in which neuronal integrity is threatened, for example when neuronal cells display decreased survival or when the neurons can no longer propagate a signal.

In one example, the administration is via injection, such as injection into the CNS (e.g., spinal cord or brain). For example, AAV6 vector or virus including the heterologous nucleic acid (such as Gsx1) may be administered near or at the site of a brain or spinal cord injury, such as rostral and/or caudal to the injury site.

In one example, the AAV6 vector or virus includes a nucleic acid encoding a Gsx1 protein that has at least 80%, at least 85%, at least 90%, at least 95%, at least 98%, at least 99%, or 100% sequence identity to SEQ ID NO: 3, 6, or 10. In another example, the AAV6 vector or virus includes a Gsx1 encoding nucleic acid including at least 80%, at least 85%, at least 90%, at least 95%, at least 98%, at least 99%, or 100% sequence identity to SEQ ID NO: 1, SEQ ID NO: 2, SEQ ID NO: 4, SEQ ID NO: 5, SEQ ID NO: 8, or SEQ ID NO: 9.

In one example, the AAV6 vector or virus including the nucleic acid encoding the heterologous nucleic acid (such as Gsx1 nucleic acid) is present in a pharmaceutical composition, such as one that includes a pharmaceutically acceptable carrier, such as saline or water.

In some examples, only a single dose of the AAV6 vector or virus is administered. However, in other examples, the method includes least two separate administrations of the AAV6 vector, such as at least 2, at least 3, at least 4, at least 5, at least 10, or at least 20 separate administrations. In some examples, the at least two separate administrations are separated by at least 1 week, at least 2 weeks, at least 3 weeks, at least 4 weeks, at least 1 month, at least 2 months, at least 3 months, at least 6 months, at least 9 months, or at least one year.

In some examples, the AAV6 vector or virus (such as an AAV6 vector or virus including a nucleic acid encoding Gsx1) occurs within 30 minutes, within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the neurological disorder (such as a brain or spinal cord injury).

The disclosed methods can further include administering to the subject a therapeutically effective amount of another neurological disorder therapeutic agent.

In some examples, the method includes selecting a subject with a neurological disorder, such as a traumatic spinal cord or brain injury, or a neurodegenerative disease. These subjects can be selected for treatment with an AAV6 vector or virus including a Gsx1 coding nucleic acid molecule.

In some examples, treating a neurological disorder using the disclosed methods includes one or more of (1) decreasing inflammation, for example at or near the injury site, such as decreasing the number of infiltrated macrophages (such as a decrease of at least 5%, at least 10%, at least 15%, at least 20%, at least 50%, at least 75%, or at least 90% for example relative to no administration of the AAV6 vector or virus encoding the heterologous protein), (2) increasing the number of neural stem/progenitor cells (NSPCs) (e.g., as determined by measuring expression of nestin, Ki67, and/or Sox2), for example at or near the injury site, (for example an increase of at least 5%, at least 10%, at least 15%, at least 20%, at least 50%, at least 75%, at least 90%, at least 100%, at least 200%, at least 300%, or at least 500%, for example relative to no administration of the AAV6 vector or virus encoding the heterologous protein), (3) increasing differentiation of NSPCs towards a specific neuronal linage, such as an increase in the number of glutamatergic neurons, increase in the number of cholinergic neurons (e.g., as determined by measuring expression of NeuN, ChAT, and/or Glut2) (for example an increase of at least 5%, at least 10%, at least 15%, at least 20%, at least 50%, at least 75%, at least 90%, at least 100%, at least 200%, at least 300%, or at least 600%, for example relative to no administration of the AAV6 vector or virus encoding the heterologous protein), decrease the number of GABAergic interneurons (e.g., as determined by measuring expression of GABA) (such as a decrease of at least 5%, at least 10%, at least 15%, at least 20%, at least 50%, at least 75%, or at least 90% for example relative to no administration of the AAV6 vector or virus encoding the heterologous protein), or combinations thereof, for example at or near the injury site, (4) decreasing astrogliosis and glial scar formation, for example at or near the injury site, such as decreasing the number of astrocytes (e.g., as determined by measuring expression of GFAP, Serpina3n, and/or CSPG) (such as a decrease of at least 5%, at least 10%, at least 15%, at least 20%, at least 50%, at least 75%, or at least 90% for example relative to no administration of the AAV6 vector or virus encoding the heterologous protein), (5) increasing locomotion of the subject (e.g., as determined by Basso Mouse Scale (BMS) score or Functional Independence Measure (FIM)) (for example an increase of at least 5%, at least 10%, at least 15%, at least 20%, at least 50%, at least 75%, at least 90%, at least 100%, at least 200%, at least 300%, or at least 600%, for example relative to no administration of the AAV6 vector or virus encoding the heterologous protein), (6) decreasing cell death, for example at or near the injury site, such as decreasing the number of cleaved caspase3 positive cells (such as a decrease of at least 5%, at least 10%, at least 15%, at least 20%, at least 50%, at least 75%, or at least 90% for example relative to no administration of the AAV6 vector or virus encoding the heterologous protein), and (7) increasing neurogenesis, axon growth, and/or axon guidance, for example at or near the injury site, (e.g., as determined by Ctnna1 and/or Col6a2 expression, Netrin signaling, expression of axonal guidance genes, and/or CREB signaling) (such as an increase of at least 5%, at least 10%, at least 15%, at least 20%, at least 50%, at least 75%, at least 90%, at least 100%, at least 200%, at least 300%, or at least 500%, for example relative to no administration of the AAV6 vector or virus encoding the heterologous protein).

In some examples, such responses are achieved within about 3 days, within about 1 week, within about 2 weeks, within about 4 weeks, within about 8 weeks, within about 12 weeks, with in about 4 months, within about 6 months, or within about 52 weeks following treatment. In some embodiments, the disclosed methods include measuring inflammation, cell proliferation, astrogliosis, glial scaring, neurogenesis, NSPC activation, and/or cell death, for example at or near an injury site, before and/or after treating a subject. In some examples, the disclosed methods include measuring locomotion of the subject before and after treating a subject.

In some embodiments, the disclosed methods include measuring locomotion before and/or after treating a subject. For example, functional outcome after spinal cord injury in humans, can be determined or measured using the Modified Barthel Index (MBI), Functional Independence Measure (FIM), Quadriplegia Index of Function (QIF), and/or the Spinal Cord Independence Measure (SCIM). Examples of such methods are described in Furlan et al., Journal of Neurotrauma. 2011; 28 (8): 1413-1430; Chumney et al., (2010). The Journal of Rehabilitation Research and Development. 47 (1): 17-30; Ota et al., Spinal Cord. 1996; 34 (9): 531-5; and Functional Recovery Outcome Measures Work Group: Anderson et al., The Journal of Spinal Cord Medicine. 2008; 31 (2): 133-144.

In some examples, an amount of (1) inflammation, for example at or near the injury site, (2) proliferation of NSPCs, for example at or near the injury site, (3) differentiation of NSPCs towards a neuronal linage, such as glutamatergic neurons, for example at or near the injury site, (4) astrogliosis and glial scar formation, for example at or near the injury site, (5) locomotion of the subject, and/or (6) cell death, for example at or near the injury site, is compared to a control. In some embodiments, the control is a value obtained prior to treatment. In some embodiments, the control is a historical control or standard reference value or range of values (such as a previously tested control sample, such as a group of subjects with or without a neurological disorder). In further examples, the control is a reference value, such as a standard value obtained from a population of normal individuals, or individual known to have a neurological disorder (such as a SCI or TBI). Similar to a control population, the value obtained from the treated subject can be compared to the mean reference value or to a range of reference values (such as the high and low values in the reference group or the 95% confidence interval). In other examples, the control is the subject (or group of subjects) treated with placebo compared to the same subject (or group of subjects) treated with the AAV6 vector or virus in a cross-over study. In further examples, the control is the subject (or group of subjects) prior to treatment.

### A. Gsx1 Proteins

In some examples, the methods provided herein utilize an AAV6 vector including a nucleic acid encoding a Gsx1 protein. Exemplary full-length Gsx1 proteins are shown in SEQ ID NOs: 3 (mouse), 6 (human), and 10 (rat). In some examples, a Gsx1 protein includes or consists of the protein sequence comprising at least 80%, at least 85%, at least 90%, at least 95%, at least 98%, at least 99%, or 100% sequence identity to SEQ ID NO: 3, 6, or 10.

In some examples, the AAV6 vector includes a nucleic acid encoding human Gsx1, such as SEQ ID NO: 6, mouse Gsx1, such as SEQ ID NO: 3, or rat Gsx1, such as SEQ ID NO: 10. Native or variant Gsx1 proteins can be used. In one example, variant Gsx1 proteins are produced by manipulating a Gsx1 nucleotide sequence. In some examples a variant Gsx1 sequence is used, such as one including amino acid substitutions, additions, deletions, or combinations thereof, as long as the protein retains the ability to increase neurogenesis, reduce astrogliosis and glial scar formation, and/or increase locomotion following spinal cord injury. Methods of measuring neurogenesis, astrogliosis and glial scar formation, and locomotion are described herein. Regions of Gsx1 that are more likely to tolerate substitution can be determined by aligning sequences (e.g., SEQ ID NOS: 3, 6, and 10), wherein amino acids conserved between species are less likely to tolerate substitutions, while amino acids that vary at a particular position are more likely to tolerate substitutions.

Variant Gsx1 proteins, such as variants of SEQ ID NOS: 3, 6, and 10, can contain one or more mutations, such as a single insertion, a single deletion, a single substitution. In some examples, the mutant Gsx1 protein includes 1-20 insertions, 1-20 deletions, 1-20 substitutions, or any combination thereof (e.g., single insertion together with 1-19 substitutions). In some examples, the variant Gsx1 protein (e.g., SEQ ID NO: 3, 6, or 10) has 1, 2, 3, 4, 5, 6, 7, 8, 9, 10, 11, 12, 13, 14, 15, 16, 17, 18, 19 or 20 amino acid changes. In some examples, SEQ ID NO: 3, 6, or 10 has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes, such as 1-8 insertions, 1-15 deletions, 1-10 substitutions, or any combination thereof (e.g., 1-15, 1-4, or 1-5 amino acid deletions together with 1-10, 1-5 or 1-7 amino acid substitutions). One type of modification or mutation includes the substitution of amino acids for amino acid residues having a similar biochemical property, that is, a conservative substitution (such as 1-4, 1-8, 1-10, or 1-20 conservative substitutions). Typically, conservative substitutions have little to no impact on the activity of a resulting peptide. For example, a conservative substitution is an amino acid substitution in SEQ ID NO: 3, 6, or 10 that does not substantially affect the ability of the Gsx1 protein to increase neurogenesis, reduce astrogliosis and glial scar formation, and/or increase locomotion following spinal cord injury, in a mammal. An alanine scan can be used to identify which amino acid residues in a Gsx1 protein, such as SEQ ID NO: 3, 6, or 10, can tolerate an amino acid substitution. In one example, these activities of Gsx1, (e.g., SEQ ID NO: 3, 6, or 10), are not altered by more than 25%, for example not more than 20%, for example not more than 10%, when an alanine, or other conservative amino acid, is substituted for 1-4, 1-8, 1-10, or 1-20 native amino acids. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions or additions) can be assessed by analyzing the function of the Gsx1 protein, such as SEQ ID NO: 2 or 4, by analyzing the ability of the variant Gsx1 protein to increase neurogenesis, reduce astrogliosis and glial scar formation, and increase locomotion following spinal cord injury, in a mammal.

### B. Gsx1 Nucleic Acid Molecules

Exemplary Gsx1 coding sequences are shown in SEQ ID NOs: 1 and 2 (mouse), SEQ ID NOs: 4 and 5 (human), and SEQ ID NOs: 8 and 9 (rat). In some examples, a Gsx1 nucleic acid molecule includes or consists of the sequence of SEQ ID NO: 1, SEQ ID NO: 2, SEQ ID NO: 4, SEQ ID NO: 5, SEQ ID NO: 8, or SEQ ID NO: 9. In some examples, a Gsx1 nucleic acid molecule encodes the protein of SEQ ID NO: 3, SEQ ID NO: 6, SEQ ID NO: 10, or a variant thereof (such as those described above). In some examples, a Gsx1 nucleic acid sequence includes or consists of the sequence of SEQ ID NO: 1, SEQ ID NO: 2, SEQ ID NO: 4, or SEQ ID NO: 5, which in some examples is part of an AAV6 vector, and in some examples operably linked to a promoter (such as a constitutive or CNS-specific promoter).

Based on the genetic code, nucleic acid sequences coding for any Gsx1 protein (e.g., SEQ ID NO: 3, SEQ ID NO: 6, or SEQ ID NO: 10), can be generated. In one example, the nucleic acid molecule encoding a Gsx1 protein comprises or consists of SEQ ID NO: 1, SEQ ID NO: 2, SEQ ID NO: 4, SEQ ID NO: 5, SEQ ID NO: 8, or SEQ ID NO: 9. Also provided are cells, plasmids and viral vectors including such nucleic acids, which can also include a promoter operably linked to the Gsx1 coding sequence.

In one example, a nucleic acid sequence that encodes a Gsx1 protein has at least 60%, at least 70%, at least 75%, at least 80%, at least 90%, at least 92%, at least 95%, at least 96%, at least 97%, at least 99% or at least 99% sequence identity to SEQ ID NO: 1, SEQ ID NO: 2, SEQ ID NO: 4, SEQ ID NO: 5, SEQ ID NO: 8, or SEQ ID NO: 9. Such sequences can readily be produced, using the amino acid sequences provided herein and that are publicly available, and the genetic code. In addition, one of ordinary skill can readily construct a variety of clones containing functionally equivalent nucleic acids, such as nucleic acids which differ in sequence but which encode the same Gsx1 protein sequence.

Nucleic acid molecules include DNA, cDNA, mRNA, and RNA sequences which encode a Gsx1 protein. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3rd Edition, W. H. 5 Freeman and Co., NY).

Codon preferences and codon usage tables for a particular species can be used to engineer isolated nucleic acid molecules encoding a Gsx1 protein (such as one encoding a protein having at least 90%, at least 95%, at least 96%, at least 97%, at least 98%, at least 99% or 100% sequence identity to SEQ ID NO: 3, 6, 10) that take advantage of the codon usage preferences of that particular species. For example, the Gsx1 proteins used in the disclosed methods can be designed to have codons that are preferentially used by a particular organism of interest (such as a human or mouse).

A nucleic acid encoding a Gsx1 protein (such as a nucleic acid molecule having at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9, or encoding a protein having at least 90%, at least 95%, at least 96%, at least 97%, at least 98%, at least 99% or 100% sequence identity to SEQ ID NO: 3, SEQ ID NO: 6, or SEQ ID NO: 10) can be cloned or amplified by in vitro methods, such as the polymerase chain reaction (PCR), the ligase chain reaction (LCR), the transcription-based amplification system (TAS), the self-sustained sequence replication system (3SR) and the QB replicase amplification system (QB). In addition, nucleic acids encoding a Gsx1 protein (such as a nucleic acid molecule having at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9, or encoding a protein having at least 90%, at least 95%, at least 96%, at least 97%, at least 98%, at least 99% or 100% sequence identity to SEQ ID NO: 3, SEQ ID NO: 6, or SEQ ID NO: 10) can be prepared by cloning techniques known to one of ordinary skill.

Nucleic acid sequences encoding a Gsx1 protein (such as a nucleic acid molecule having at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9, or encoding a protein having at least 90%, at least 95%, at least 96%, at least 97%, at least 98%, at least 99% or 100% sequence identity to SEQ ID NO: 3, SEQ ID NO: 6, or SEQ ID NO: 10) can be prepared by any suitable method including, for example, cloning of appropriate sequences or by direct chemical synthesis by methods such as the phosphotriester method; the phosphodiester method; the diethylphosphoramidite method; the solid phase phosphoramidite triester method, for example, using an automated synthesizer; and, the solid support method. Chemical synthesis produces a single stranded oligonucleotide. This can be converted into double stranded DNA by hybridization with a complementary sequence, or by polymerization with a DNA polymerase using the single strand as a template. While chemical synthesis of DNA is generally limited to sequences of about 100 bases, longer sequences may be obtained by the ligation of shorter sequences.

In one example, an AAV6 vector or virus encoding a Gsx1 protein (such as one having at least 90%, at least 95%, at least 96%, at least 97%, at least 98%, at least 99% or 100% sequence identity to SEQ ID NO: 3, SEQ ID NO: 6, or SEQ ID NO: 10) is prepared by inserting a cDNA which encodes a Gsx1 protein (such as a nucleic acid molecule having at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9) into a vector including AAV6 flanking ITRs. The insertion can be made so that the Gsx1 protein is read in frame so that the Gsx1 protein is produced.

### C. Pharmaceutical Compositions and Dosing

Pharmaceutical compositions that include an AAV6 vector or virus including a nucleic acid encoding a Gsx1 protein (such as a nucleic acid molecule having at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9, or a nucleic acid molecule that encodes a protein having at least 90%, at least 95%, at least 96%, at least 97%, at least 98%, at least 99% or 100% sequence identity to SEQ ID NO: 3, SEQ ID NO: 6, or SEQ ID NO: 10), can be formulated with an appropriate pharmaceutically acceptable carrier (such as water or saline), depending upon the particular mode of administration chosen. Such compositions can be administered to a subject with a neurological disorder using the disclosed methods. In one example, the pharmaceutical composition is suitable for injection, such as injection into the CNS, for example at or near the site of injury (e.g., rostral and/or caudal to the injury site). In some examples, intraparenchymal, introcerebroventricular, or intrathecal (cisternal and lumbar) injections are used to target brain and/or spinal cord.

In some examples, the pharmaceutical composition consists essentially of an AAV6 vector or virus encoding a Gsx1 protein (such as a protein having at least 90%, at least 95%, at least 96%, at least 97%, at least 98%, at least 99% or 100% sequence identity to SEQ ID NO: 3, SEQ ID NO: 6, or SEQ ID NO: 10) and a pharmaceutically acceptable carrier. In such examples, additional therapeutically effective agents are not included in the composition.

In other examples, the pharmaceutical composition includes an AAV6 vector or virus encoding a Gsx1 protein (such as a protein having at least 90%, at least 95%, at least 96%, at least 97%, at least 98%, at least 99% or 100% sequence identity to SEQ ID NO: 3, SEQ ID NO: 6, or SEQ ID NO: 10) and a pharmaceutically acceptable carrier. Additional therapeutic agents, such as agents for the treatment of a neurological disorder (such as SCI, TBI or neurodegenerative disorder), can be included. Thus, the pharmaceutical compositions can include a therapeutically effective amount of another agent. Examples of such agents include, without limitation, those listed below, or combinations thereof.

Pharmaceutically acceptable carriers and excipients useful in this disclosure are known to one of ordinary skill in the art. See, e.g., Remington: The Science and Practice of Pharmacy, Adejare (Ed.), Academic Press, London, United Kingdom, 23^{rd} Edition (2021). For instance, formulations usually include injectable fluids that are pharmaceutically and physiologically acceptable fluid vehicles such as water, physiological saline, other balanced salt solutions, aqueous dextrose, glycerol or the like. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some examples, an AAV6 vector encoding a Gsx1 protein (such as a protein having at least 90%, at least 95%, at least 96%, at least 97%, at least 98%, at least 99% or 100% sequence identity to SEQ ID NO: 3, SEQ ID NO: 6, or SEQ ID NO: 10) is included in a controlled release formulation, for example, a microencapsulated formulation. Various types of biodegradable and biocompatible polymers, methods can be used, and methods of encapsulating a variety of synthetic compounds, proteins and nucleic acids can be used (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other examples, an AAV6 vector or virus encoding a Gsx1 protein (such as a protein having at least 90%, at least 95%, at least 96%, at least 97%, at least 98%, at least 99% or 100% sequence identity to SEQ ID NO: 3, SEQ ID NO: 6, or SEQ ID NO: 10) is included in a nanodispersion system. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers that can be used include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

Many types of release delivery systems can be used. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an AAV6 vector including a nucleic acid molecule that encodes a protein having at least 90%, at least 95%, at least 96%, at least 97%, at least 98%, at least 99% or 100% sequence identity to SEQ ID NO: 3, SEQ ID NO: 6, or SEQ ID NO: 10), is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

A long-term sustained release implant can be suitable for treatment of chronic conditions, such as neurological disorders. Long-term release, as used herein, means that the implant is constructed and arranged to deliver therapeutic levels of the active ingredient for at least 30 days, or at least 60 days. Long-term sustained release implants include some of the release systems described above. These systems have been described for use with nucleic acids (see U.S. Pat. No. 6,218,371). For use in vivo, nucleic acids and peptides are relatively resistant to degradation (such as via endo- and exo-nucleases).

The dosage form of the pharmaceutical composition can be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate.

In some examples, the amount of AAV6 vector or virus including a Gsx1 coding sequence (such as encoding a protein having at least 90%, at least 95%, at least 96%, at least 97%, at least 98%, at least 99% or 100% sequence identity to SEQ ID NO: 3, SEQ ID NO: 6, or SEQ ID NO: 10) is an amount that (1) decreases inflammation, for example at or near the injury site, such as decrease the number of infiltrated macrophages (such as a decrease of at least 5%, at least 10%, at least 15%, at least 20%, at least 50%, at least 75%, or at least 90% for example relative to no administration of AAV6 vector or virus including a Gsx1 coding sequence), (2) increases the number of neural stem/progenitor cells (NSPCs) (e.g., as determined by measuring expression of nestin, and/or doublecortin), for example at or near the injury site, (for example an increase of at least 5%, at least 10%, at least 15%, at least 20%, at least 50%, at least 75%, at least 90%, or at least 100%, for example relative to no administration of AAV6 vector or virus including a Gsx1 coding sequence), (3) increases differentiation of NSPCs towards a neuronal linage, such as an increase in the number of glutamatergic neurons and cholinergic neurons (and decrease in the number of GABAergic interneurons) for example at or near the injury site, (e.g., as determined by measuring expression of NeuN, ChAT, and/or Glut2) (for example an increase of at least 5%, at least 10%, at least 15%, at least 20%, at least 50%, at least 75%, at least 90%, at least 100%, at least 200%, at least 300%, or at least 600%, for example relative to no administration of AAV6 vector or virus including a Gsx1 coding sequence), decreases the number of GABAergic interneurons (e.g., as determined by measuring expression of GABA) (for example a decrease of at least 5%, at least 10%, at least 15%, at least 20%, at least 50%, at least 75%, or at least 90% for example relative to no administration of AAV6 vector or virus including a Gsx1 coding sequence), (4) decreases astrogliosis and glial scar formation, for example at or near the injury site, such as decreasing the number of astrocytes (e.g., as determined by measuring expression of GFAP, Serpina3n, and/or CSPG) (such as a decrease of at least 5%, at least 10%, at least 15%, at least 20%, at least 50%, at least 75%, or at least 90% for example relative to no administration of AAV6 vector or virus including a Gsx1 coding sequence), (5) increases locomotion of the subject (e.g., as determined by Basso Mouse Scale (BMS) score or Functional Independence Measure (FIM)) (for example an increase of at least 5%, at least 10%, at least 15%, at least 20%, at least 50%, at least 75%, at least 90%, or at least 100%, at least 200%, at least 300%, or at least 600%, for example relative to no administration of AAV6 vector or virus including a Gsx1 coding sequence), and/or (6) decreases cell death, for example at or near the injury site, such as decrease the number of cleaved caspase3 positive cells (such as a decrease of at least 5%, at least 10%, at least 15%, at least 20%, at least 50%, at least 75%, or at least 90% for example relative to no administration of AAV6 vector or virus including a Gsx1 coding sequence).

The pharmaceutical compositions that include an AAV6 vector including a Gsx1 coding sequence (such as one encoding a protein having at least 90%, at least 95%, at least 96%, at least 97%, at least 98%, at least 99% or 100% sequence identity to SEQ ID NO: 3, SEQ ID NO: 6, or SEQ ID NO: 10, such as a nucleic acid molecule having at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9) can be formulated in unit dosage form, suitable for individual administration of precise dosages. Generally, the quantity of recombinant viral vector, carrying the nucleic acid coding sequence of Gsx1 protein to be administered, is based on the titer of virus particles. In one non-limiting example, for example when an AAV6 vector or virus is utilized for administration of a nucleic acid encoding an Gsx1 protein (such as a protein having at least 90%, at least 95%, at least 96%, at least 97%, at least 98%, at least 99% or 100% sequence identity to SEQ ID NO: 3, SEQ ID NO: 6, or SEQ ID NO: 10, such as a vector or virus containing a nucleic acid molecule having at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9), a unit dosage (e.g., 0.5-5 μl) contains about 10⁵ to about 10¹⁰ plaque forming units (pfu)/ml. Thus, in some examples, the recipient subject is administered a dose of about 10⁵ to about 10¹⁰ pfu/ml of recombinant virus in the composition. In some examples, the recipient subject is administered a dose of at least 10⁵ pfu/ml, at least 10⁶ pfu/ml, at least 10⁷ pfu/ml, at least 10⁸ pfu/ml, at least 10⁹ pfu/ml, or at least 10¹⁰ pfu/ml.

In some examples, a disclosed AAV6 vector or virus including a Gsx1 coding sequence is administered at a dose of about 10⁴ to about 10¹⁴ virions (viral particles). In some examples, the AAV6 including a Gsx1 coding sequence is administered at a dose of about 10⁵ to about 10¹³ virions or about 10⁸ to about 10¹² virions. In specific non-limiting examples, the AAV6 including a Gsx1 coding sequence is administered at a dose of at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, or at least about 1×10¹⁴ virions. In other non-limiting examples, the AAV6 including a Gsx1 coding sequence is administered at a dose of no more than about 10¹⁰, no more than about 10¹¹, no more than about 10¹², no more than about 10¹³, or no more than about 10¹⁴ virions. In additional examples, the AAV6 including a Gsx1 coding sequence is administered at a dose of about 10⁵ to about 10¹⁴ vector genomes (vg) (such as about 1.6×10⁹ vg, about 5×10⁹ vg, about 1×10¹⁰ vg, about 5×10¹⁰ vg, about 1×10¹¹ vg, about 5×10¹¹ vg, about 1×10¹² vg, about 5×10¹² vg, about 1×10¹³ vg, about 5×10¹³ vg or about 1×10¹⁴ vg. In some examples, the AAV6 including a Gsx1 coding sequence is administered at a dose of about 10¹⁰ to about 10¹² vgs, such as about 10¹¹ vgs.

In other embodiments, the rAAV is administered at a dose of about 1×10¹⁰ to about 1×10¹⁴ vector genomes (vg)/kg. In some examples, the rAAV is administered at a dose of about 1×10¹⁰ to about 1×10¹⁵ vg/kg. In specific non-limiting examples, the rAAV is administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In other non-limiting examples, the rAAV is administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In one non-limiting example, the rAAV is administered at a dose of about 1×10¹² vg/kg, about 4×10¹² vg/kg, or about 1×10¹³ vg/kg.

Examples of methods for administering the composition into mammals include, but are not limited to, injection of the composition into the affected tissue (such as into the brain or spinal cord) or intravenous, subcutaneous, intradermal or intramuscular administration of the virus. In some examples, the subject receives the unit dosage in an injection at a single site, while in other examples, the subject receives the unit dosage in a divided form with injection at multiple sites in the affected tissue.

The compositions of this disclosure can be administered to humans or other animals by any means, including orally, intravenously, intramuscularly, intraperitoneally, intranasally, intradermally, intraparenchymally, introcerebroventricularly, intrathecally (e.g., cisternal and lumbar), subcutaneously, via inhalation or via suppository. In one non-limiting example, the composition is administered via injection. In some examples, site-specific administration of the composition can be used, for example by administering the AAV6 vector or virus including the Gsx1 coding sequence to CNS tissue (for example the brain or spinal cord, for example at or near the area of injury, such as rostral and/or caudal to the injury site). In some examples, administration is an intrathecal injection (e.g., of AAV6 vector or virus including the Gsx1 coding sequence) to treat SCI in lumbar/sacral region, a cisterna magna injection (e.g., of AAV6 vector or virus including the Gsx1 coding sequence) to treat SCI in cervical/thoracic region, or intraparenchymal or introcerebroventricular injection (e.g., of AAV6 vector or virus including the Gsx1 coding sequence) to treat traumatic brain injury.

Treatment can involve a single administration, or multiple administrations (such as at least two separate administrations), such as doses over a period of a few days to months, or even years. For example, an AAV6 vector or virus including Gsx1 coding sequence (such as encoding a protein having at least 90%, at least 95%, at least 96%, at least 97%, at least 98%, at least 99% or 100% sequence identity to SEQ ID NO: 3, SEQ ID NO: 6, or SEQ ID NO: 10), can be administered in a single dose, or in several doses, for example daily, weekly, monthly, or yearly, during a course of treatment. In a particular non-limiting example, treatment involves administration once monthly, once yearly, or every-other-month. In some examples, where multiple doses are administered, the at least two separate administrations can be separated by at least 1 week, at least 2 weeks, at least 3 weeks, at least 4 weeks, at least 1 month, at least 2 months, at least 3 months, at least 6 months, at least 9 months, or at least one year. In some examples, the first dose (and in some examples only dose) administrated occurs within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the neurological disorder, such as within 1 to 24 hours, 2 to 24 hours, 4 to 24 hours, or 1 to 96 hours of the onset of the neurological disorder.

### D. Administration of Additional Therapy

In some examples, a disclosed composition is administered in combination (such as sequentially, simultaneously, or contemporaneously) with one or more other agents, such as those useful in the treatment of a neurological disorder. The term “administration in combination” or “co-administration” refers to both concurrent and sequential administration of the active agents.

In some examples, the composition is administered to a subject with a traumatic spinal cord or brain injury in combination with effective doses of one or more of stem cells, steroids (e.g., methylprednisolone), and intravenous fluids. In some examples, the subject also receives surgery, hypothermia treatment, or both. Administration of a an AAV6 vector or virus including Gsx1 coding sequence, may also be in combination with lifestyle modifications, such as increased physical activity, physical therapy, or immobilization (e.g., in a hard collar).

In some examples, an AAV6 vector or virus including a Gsx1 coding sequence is administered to a subject with a neurological disorder of the brain, such as Parkinson's disease, Alzheimer's disease, stroke (ischemic or hemorrhagic), ischemia, epilepsy, Huntington's disease, multiple sclerosis, amyotrophic lateral sclerosis, in combination with effective doses of one or more other therapeutic agents. For example, if the subject has Parkinson's disease, the method can further include administering a therapeutically effective amount of one or more of stem cells, deep brain stimulation, surgery (e.g., pallidotomy or thalamotomy) benztropine mesylate (Cogentin), entacapone (Comtan), dopar, dopamine agonist (e.g., apomorphine (Apokyn), pramipexole (Mirapex), ropinirole HCl (Requip), and rotigotine (Neupro)), larodopa, levodopa and carbidopa (Sinemet), rasagiline (Azilect), safinamide (Xadago), tasmar and trihexphenidyl (Artane). For example, if the subject has Alzheimer's disease, the method can further include administering a therapeutically effective amount of one or more of stem cells, a cholinesterase inhibitor (e.g., Razadyne® (galantamine), Exelon® (rivastigmine), or Aricept® (donepezil)), an N-methyl D-aspartate (NMDA) antagonist (e.g., memantine), Celexa® (citalopram), Remeron® (mirtazapine), Zoloft® (sertraline), Wellbutrin® (bupropion), Cymbalta® (duloxetine), and Tofranil® (imipramine). For example, if the subject has had a stroke, the method can further include administering a therapeutically effective amount of one or more of a tissue plasminogen activator (e.g., Alteplase IV r-tPA) for an ischemic stroke, or surgery (e.g., install a coil or clip to stop blood loss) for a hemorrhagic stroke. For example, if the subject has ischemia (e.g., cardiac ischemia or mesenteric artery ischemia), the method can further include administering a therapeutically effective amount of one or more of a vasodilator, anticoagulant, (e.g., heparin, aspirin), nitrate, ACE inhibitor, ranolazine, and surgery. For example, if the subject has epilepsy, the method can further include administering a therapeutically effective amount of one or more of an anti-seizure or anti-epileptic medication (e.g., carbamazepine, valproate, lamotrigine, dilantin or phenytek, ohenobarbital, tegretol or Carbatrol, mysoline, zarontin, depakene, depakote, depakote ER, valium and similar tranquilizers such as Tranxene and Klonopin, felbatol, gabitril, keppra, lamictal, lyrica, neurontin, topamax, trileptal, and, zonegran), surgery, vagus nerve stimulation, deep brain stimulation, and a ketogenic diet. For example, if the subject has Huntington's disease, the method can further include administering a therapeutically effective amount of one or more of a monoamine depleter (e.g., tetrabenazine or amantadine), SSRI antidepressant (e.g., fluoxetine citalopram, paroxetine, and sertraline) or other anti-depressant (e.g., amitriptyline, mirtazapine, duloxetine, and venlafaxine), antipsychotic drug (e.g., quetiapine, risperidone or olanzapine), mood-stabilizing drug (e.g., valproate or carbamazepine), and a high protein diet. For example, if the subject has multiple sclerosis, the method can further include administering a therapeutically effective amount of one or more of stem cells, a corticosteroid (e.g., methylprednisolone or prednisone), an interferon beta blocker (e.g., copaxone, teriflunomide, or mitoxantrone).

For example, if the subject has amyotrophic lateral sclerosis (ALS), the method can further include administering a therapeutically effective amount of one or more of a glutamate antagonist (e.g., riluzole) and a neuroprotective agent (e.g., edaravone). Thus, in some examples, the pharmaceutical composition that includes an AAV6 vector or virus including a Gsx1 coding sequence further includes one or more of these therapeutic agents. Administration of an AAV6 vector or virus including Gsx1 coding sequence, may also be in combination with increased physical activity, speech or language therapy, occupational therapy, physical therapy, or combinations thereof.

### EXAMPLES

The following examples are provided to illustrate particular features of certain aspects of the disclosure, but the scope of the claims should not be limited to those features exemplified.

### Example 1

### Materials and Methods

This example describes Materials and Methods used for Examples 2-8.

AAV and LV constructs: Viral constructs: ssAAV5-CMV-eGFP, ssAAV6-CMV-eGFP, ssAAVrh10-CMV-eGFP, ssAAV6-CMV-eGFP, and scAAV6-CMV-Gsx1 were manufactured by Vector Biolabs (Malvern, PA). LV-CMV-eGFP and LV-CMV-Gsx1-SV40-eGFP were manufactured by Applied Biological Materials Inc. (Richmond, BC, Canada).

Rat model of lateral hemisection SCI: Male Sprague Dawley rats (8-12-week-old) were purchased from Charles River Laboratories. Rats were acclimated to the animal facility for 1 week. Rats were anesthetized with 3% Isoflurane and maintained at 2% Isoflurane, then placed on heating pad set to low. Eye lubricant was applied, the surgical site shaved, and sterilized using betadine and 70% ethanol solutions. Analgesics were administered including buprenorphine SR and bupivacaine 0.125%. An incision was made with 10 blade scalpel between cervical and lumbar spinal level. The muscle was dissected using surgical microscissors and remove the dorsal process of thoracic vertebrae 9 (T9) and T10 were removed with bone rongeur to expose the spinal cord. A clamp was applied to the surrounding muscle and a lateral hemisection spinal cord injury was generated via surgical microscissors. 1.5 μL virus treatment was injected in the BSL2 facility at 500 nL/min using 10 μL Hamilton syringe at 1.0 mm rostral/caudal to injury site. A volume of 0.5 μL virus treatment was injected at depths: 0.5 mm, 1.0 mm, 1.5 mm to ensure total 3.0 μL virus penetrates throughout the injured spinal cord. Adipose tissue from the nape of neck was removed and placed on the exposed T9-10 spinal cord injury site. Two 3-0 sutures were applied to close the muscle and fat adjacent to the laminectomy. Wound clips were applied, and the animal was placed in recovery cage on heating pad and observed until awake and alert. Sterile saline was administered throughout the surgery to ensure animal hydration and cefazolin antibiotic was administered immediately after the surgery. Food and water were provided ad libitum.

Animals were monitored daily for pain, distress, hydration, and surgical site infection. Animal bladders expressed twice daily and administered 1.0 mL bolus saline and cefazolin antibiotic daily for the duration of the study. Bladder infections were treated with enrofloxacin and autophagia was treated with acetaminophen as needed. All procedures were carried out under protocols approved by the Rutgers University Institutional Animal Care and Use Committee and conformed to NIH guidelines.

Rat model of contusion SCI: Female Sprague Dawley rats (8-12-week-old) were anesthetized with isoflurane (2.5%) before performing a laminectomy to remove the dorsal process of thoracic vertebrae 9 (T9) and expose the spinal cord. The lateral processes of T8 and T10 were clamped and a 200 kDyn injury was induced using the Infinite Horizon Impactor (Precision Systems & Instrumentation). Body temperature was monitored and maintained throughout the surgery using a thermo-regulated heating pad. Following injury, animals received viral treatment: AAV6-GFP, AAV-Gsx1, or LV-Gsx1-GFP via stereotaxic injection into the 4 corners of injury site in the BSL2 facility. After injection, muscle layers were sutured (Ethicon) and skin was closed using wound clips and analgesics, ringer lactate, antibiotics were administered, and returned to the hazard room facility for postoperative care. Animals were housed in temperature-controlled incubators until normothermic and then placed in cages on temperature regulated heating pads in a recovery area. Animals were housed in pairs in standard plastic cages. Food and water were provided ad libitum. Buprenorphine (0.05 mg/kg) was administered twice-a-day for the first three days post-surgery to alleviate pain. Lactated Ringer's solution (10 ml) was provided 1-2 times per day for the first three days post-surgery to prevent dehydration. Gentamycin (5 mg/kg) was administered once daily for the first 7 days post-surgery to prevent infections.

Contusion surgeries, animal care, locomotor and bladder function analysis, and euthanasia were performed by the Burke Neurological Institute at Weill Cornell Medicine (White Plains, NY). All procedures were carried out under protocols approved by the Weill Cornell Medicine Institutional Animal Care and Use Committee and conformed to NIH guidelines.

Locomotor and bladder function analysis: Recovery of motor function was assessed via BBB locomotor scale method. Prior to recording baseline measurements, rats were allowed to adapt to the open field and pretrained for 10 days. Pre-injury baseline values were collected on the day before SCI surgery (day 0). Following SCI and gene therapy intervention rats' ability to locomote was observed, scored, and documented on post-injury days 1, 4, 14, 35 and 56. Briefly, animals were placed on a flat surface with 6+ inch high walls and allowed to move/walk around the “pool” for 4 mins. Sham and SCI rat's joint movement, hindlimb movements, stepping, forelimb and hindlimb coordination, trunk position and stability, paw placement and tail position were monitored and scored. The scale (0-21) represents sequential recovery stages. Bladders were expressed twice daily and relative volume was measured manually.

Tissue processing, sectioning, and immunohistochemistry (IHC): Animals were anesthetized with 3% isoflurane and placed on dissection tray. An incision was made in the mid-abdomen and the diaphragm dissected. Incisions on either side of the ribcage were made and the ribcage pinned above the chest. The heart was held with forceps and the right anterior vena cava cut using surgical microscissors. A safety blood collection needle was placed into the left ventricle and 15 ml standard 1× Phosphate Buffered Saline (1×PBS) was pumped at a rate of 4 ml/min into the left ventricle, followed by 15 ml 4% Paraformaldehyde (PFA) solution. Vertebral columns were removed, placed on ice in 4% PFA. An 8 mm section centered at T9-10 was dissected immediately using forceps, surgical microscissors, and bone rongeur. Rats were perfused with saline and fixed with 4% paraformaldehyde and spinal cords were collected, dissected, and cryopreserved in 30% sucrose solution.

Tissues were washed overnight in 4% PFA, then washed in 1×PBS for 1.5 hours and placed in sucrose. After 24-48 hours, tissues were saturated and submerged in optimal cutting temperature (OCT) fluid at −80 C. Tissues were sectioned using cryomicrotome, e.g., coronal, or sagittal plane, at 12 μm thickness onto charged glass slides and split into 6 major sections of the spinal cord. Sectioned tissues were stored in long term at −80° C. or short term in 4° C.

Cryosectioned tissues were removed from −80° C. and placed in room temperature for 30 minutes. Tissues were rehydrated with 1×Phosphate buffered saline (PBS) and placed into slide chamber. Methanol antigen retrieval was performed for 10 minutes and washed with 1×PBS twice for 5 minutes. Tissues were incubated with diluted primary antibody solutions (Table 1) and placed overnight at 4° C. Tissues were washed in 1×PBS three times for 10 minutes and incubated with diluted secondary antibody solutions for 60 minutes at room temperature. The tissues were then washed with 1×PBS twice for 10 minutes and incubated with diluted DAPI nuclear stain solution for 5 minutes. Tissues were washed in 1×PBS three times for 5 minutes. Slides were removed from chamber and left to dry, then mounting media and glass cover slip were applied. The Gsx1 antibody was used to evaluate virally infected cells in the SCI+AAV-Gsx1, as the virus is self-complementary and limited in size. Virus mediated Gsx1 expression was validated by IHC using anti-Gsx1 antibody (Sigma-Aldrich #SAB2104632; data not shown).

TABLE 1
List of antibodies used in this study

| | | | Host | | | Dilutio |
|---|---|---|---|---|---|---|
| Primary Antibody | Company | Product ID | Species | Type | RRID | n |
| GSX1 (N-terminal) | Sigma-Aldrich | SAB210463 | Rabbit | Polyclonal | AB_10667904 | 1:200 |
| produced in rabbit | | 2 | | | | |
| PCNA (F-2) | Santa Cruz | sc-25280 | Mouse | Monoclonal | AB_628109 | 1:100 |
| | Biotechnology | | | | | |
| Nestin (Rat) | R&D Systems | AF2736 | Goat | Polyclonal | AB_416673 | 1:100 |
| SOX2 | R&D Systems | AF2018 | Goat | Polyclonal | AB_355110 | 1:200 |
| (Human/Mouse/Rat) | | | | | | |
| NG2 | Millipore Sigma | MAB5384 | Mouse | Monoclonal | AB_177646 | 1:50 |
| β-Tubulin III | Sigma-Aldrich | T8578 | Mouse | Monoclonal | AB_1841228 | 1:100 |
| (neuronal) | | | | | | |
| Notch-1 | R&D Systems | AF1057 | Goat | Polyclonal | AB_2153372 | 1:100 |
| (Mouse/Rat) | | | | | | |
| Doublecortin (C-18) | Santa Cruz | sc-8066 | Goat | Polyclonal | AB_2088494 | 1:1000 |
| GFAP | Invitrogen | 13-0300 | Rat | Monoclonal | AB_2532994 | 1:500 |
| NeuN (clone A60) | Millipore Sigma | MAB377 | Mouse | Monoclonal | AB 2298772 | 1:200 |
| CS56 | Millipore Sigma | C8035 | Mouse | Monoclonal | AB_476879 | 1:200 |
| GABA (GB-69) | Sigma-Aldrich | SAB4200721 | Mouse | Monoclonal | AB_2891218 | 1:200 |
| VGlut2 | abcam | Ab79157 | Mouse | Monoclonal | AB_1603114 | 1:200 |
| ChAT | Sigma | AB144P | Goat | Polyclonal | AB_2079751 | 1:100 |
| 5-HT | Immunostar | 20079 | Goat | Polyclonal | AB_572262 | 1:5000 |
| Myelin Basic | Sigma-Aldrich | MAB395 | Rat | Monoclonal | AB_240845 | 1:500 |
| Protein | | | | | | |
| Synaptophysin | ProteinTech | 67864-1 | Mouse | Monoclonal | AB_2918622 | 1:500 |
| MAP2 | Antibodies.com | A85363 | Chicken | Polyclonal | AB_2748940 | 1:500 |
| Caspase-3 | Signalway | 44144 | Mouse | Monoclonal | N/A | 1:100 |
| | Antibody | | | | | |

| Secondary Antibody | Company | Product ID | Type | RRID | Dilution |
|---|---|---|---|---|---|
| Alexa Fluor 488 Donkey | Jackson Immuno | 711-545-152 | Polyclonal | AB_2313584 | 1:200 |
| anti Rabbit | Research | | | | |
| Alexa Fluor 594 Donkey | Jackson Immuno | 715-585-150 | Polyclonal | AB_2340854 | 1:200 |
| anti Mouse | Research | | | | |
| Alexa Fluor 594 Donkey | Jackson Immuno | 711-585-152 | Polyclonal | AB_2340621 | 1:200 |
| anti Rabbit | Research | | | | |
| Alexa Fluor 594 Donkey | Jackson Immuno | 712-585-153 | Polyclonal | AB_2340689 | 1:200 |
| anti Rat | Research | | | | |
| Alexa Fluor 594 Donkey | Jackson Immuno | 705-585-003 | Polyclonal | AB_2340432 | 1:200 |
| anti Goat | Research | | | | |
| Alexa Fluor 647 Donkey | Jackson Immuno | 715-605-150 | Polyclonal | AB_2340862 | 1:200 |
| anti Mouse | Research | | | | |
| Alexa Fluor 647 Donkey | Jackson Immuno | 711-605-152 | Polyclonal | AB_2492288 | 1:200 |
| anti Rabbit | Research | | | | |
| Alexa Fluor 647 Donkey | Jackson Immuno | 712-605-153 | Polyclonal | AB_2340694 | 1:200 |
| anti Rat | Research | | | | |
| Alexa Fluor 647 Donkey | Jackson Immuno | 705-605-003 | Polyclonal | AB_2340436 | 1:200 |
| anti Goat | Research | | | | |

Microscopy and image analysis: Four to six sections from each animal were analyzed. Images were captured at the same exposure, threshold, and intensity per condition using Zeiss AxioVision imager A1 (Zeiss, Germany) and Echo Revolve (San Diego, CA) at wavelengths 488, 547, 649 nm. Images were processed and cell counted using ImageJ. Co-labeled cells with viral reporter GFP and specific markers were manually counted in separate RGB channels and merged images in an area of 438 μm by 328 μm region adjacent to the injection and lesion site. Alternatively, ZVI files were converted to TIFF format using python code and TIFF files are analyzed using Ilastik's pixel classification module. Pixel intensity and area are quantified, and statistical analysis is performed. A minimum of 5-10 images per animal are required to generate data using cell counting or Ilastik analysis methods. Overall, considerations include systematic/random sampling, antibody staining clearly identifying cells or protein of interest, and calculation of total cell signal were made. Images containing artifacts, tissue folds, and non-specific or unclear antibody binding were excluded from analysis.

Statistical analysis: GraphPad Prism 6 was used for all statistical analysis. Comparisons between two individual groups were analyzed with two-tailed students T-test (α=0.05). Comparisons between three groups or more were analyzed with a one-way ANOVA and Tukey multiple comparisons test (α=0.05). BBB scores and vector biodistribution were analyzed using two-way repeated-measures ANOVA (α=0.05) with a Tukey's multiple comparisons post hoc test

### Example 2

### AAV6 Preferentially Transduces NSPCs in the Injured Rat Spinal Cord

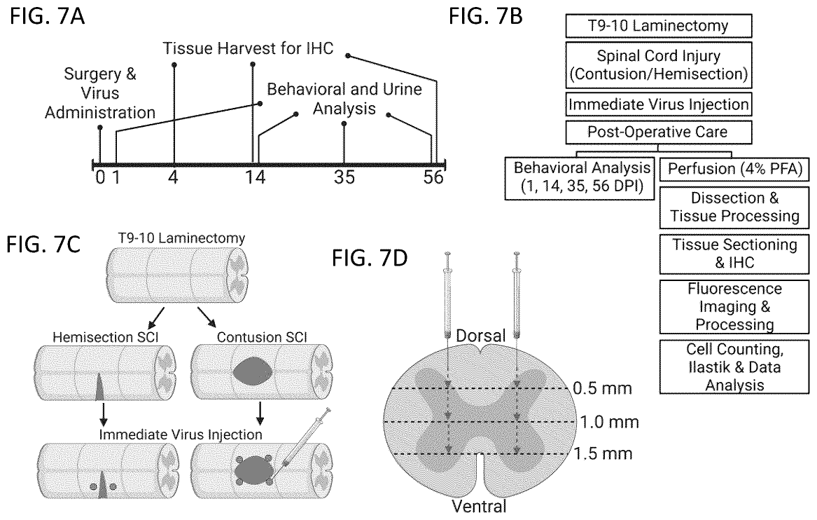

Since LV bears biosafety concerns, e.g., insertional mutagenesis, we reviewed AAV serotypes with NSPC affinity. Initially, we identified three potential serotypes: AAV5, AAV6, and AAVrh10 based on their known tropism. We then evaluated which AAV serotype transduces NSPCs with the highest efficiency. We screened the three selected candidates in a rat model of lateral hemisection SCI. Viral constructs with a ubiquitous cytomegalovirus (CMV) promoter and GFP reporter, AAV5-GFP, AAV6-GFP, and AAVrh10-GFP, were selected and tested. LV-GFP served as a positive control. A total number of 12 male Sprague Dawley rats were randomly divided into the following groups: SCI+AAV5-GFP, SCI+AAV6-GFP, SCI+AA Vrh10-GFP, SCI+LV-GFP. A total of 3.0 μl virus was injected at three depths into the spinal cord at 500 nl/min: 0.5 mm, 1.0 mm, 1.5 mm, at approximately 1.0 mm rostral and caudal to the injury site immediately following SCI (FIGS. 7A-7D). Animals were sacrificed and spinal cords were harvested in the acute stage at 4 days post-injury (4 dpi).

Immunohistochemistry (IHC) analysis was performed to quantify the expression of well-established NSPC marker Nestin. The efficiency of viral transduction was determined by the percentage of GFP+ cells among the total number of DAPI+ cells at the viral injection site adjacent to the lesion core. Transduction efficiency in NSPCs was defined as the percentage of GFP and Nestin co-labeled cells (GFP+/Nestin+) among virally infected cells (GFP+). We observed that the GFP+ cells were concentrated at the injection sites and evenly distributed throughout the injury, approximately 1 mm rostral/caudal to the lesion core (data not shown). The Nestin+ cells were concentrated near the lesion site and did not distinctly pass through the ependymal layer of the central canal (CC) into the uninjured side. However, some NSPC activation was seen on the uninjured lateral side closest to the hemisection injury (data not shown).

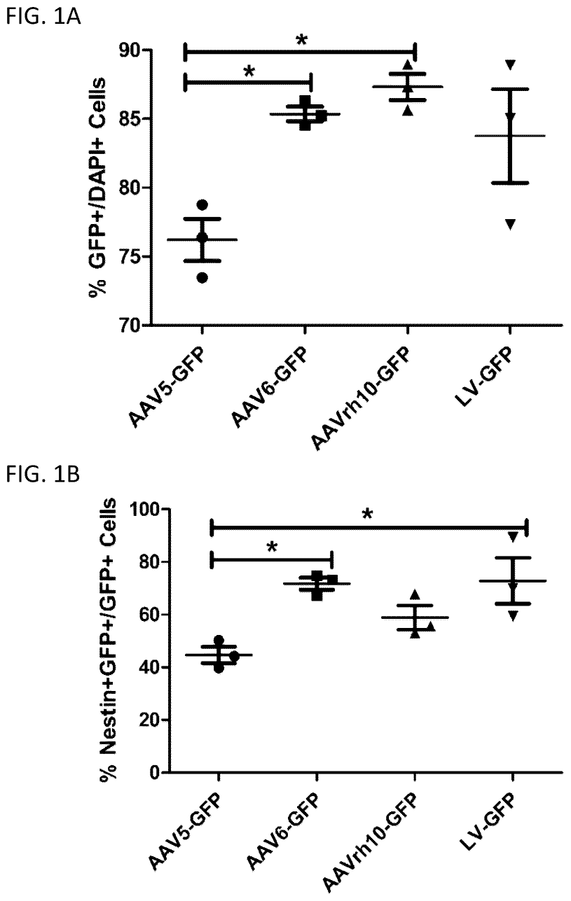

Cell count analysis showed the percentage of AAV6-GFP+ cells (85.36%+0.52; n=3) and AAVrh10-GFP+ cells (87.32%+0.95; n=3) among the total number of cells (DAPI+) in the counted area were significantly higher than that of the AAV5-GFP group (76.20%+1.53; n=3), compared with the percentage of LV-GFP (83.75%+3.40; n=3) control group. This indicates that the serotypes of AAV6 and AAVrh10 have a higher transduction efficiency than AAV5 (FIG. 1A). The percentage of GFP+ and Nestin+ cells among virally infected GFP+ cells in AAV6-GFP (71.75%+2.28; n=3) and AAVrh10-GFP (58.84%+4.59; n=3) were significantly higher than that of AAV5-GFP group (44.71%+3.07; n=3), compared with LV-GFP (72.89%+8.75; n=3) control (FIG. 1B). While no significant difference in transduced NSPCs was found between AAV6-GFP and AAVrh10-GFP, a trend and greater significant difference with AAV5-GFP infected NSPCs (FIG. 1A) indicates that AAV6 serotype has the highest transduction efficiency for NSPCs. The high transduction efficiency and NSPC specific transduction rates reflect the infected cells at the injection sites, directly overlapping with a region of high NSPC activation after SCI. Based on our findings, the NSPC specific AAV6 was selected to further test the efficacy of Gsx1 for SCI treatment in a rat model of contusion SCI.

### Example 3

### AAV6-Gsx1 Promotes NSPC Activation, Proliferation, and Neurogenesis in the Acute SCI

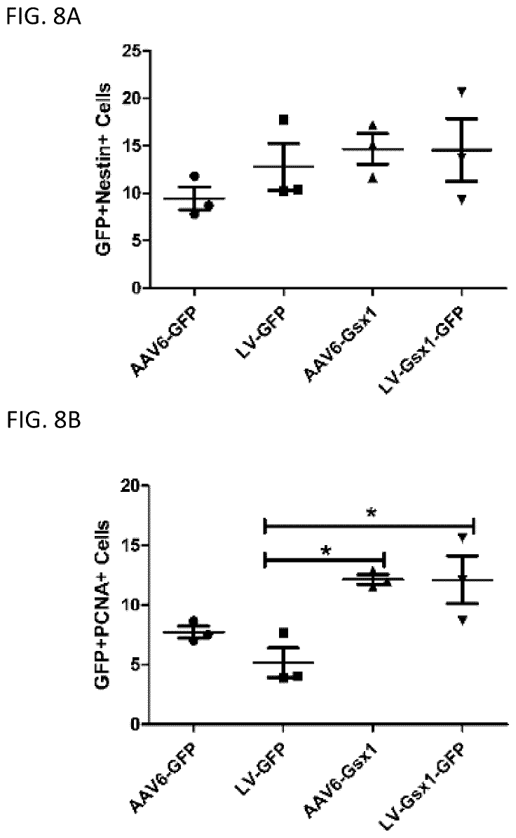

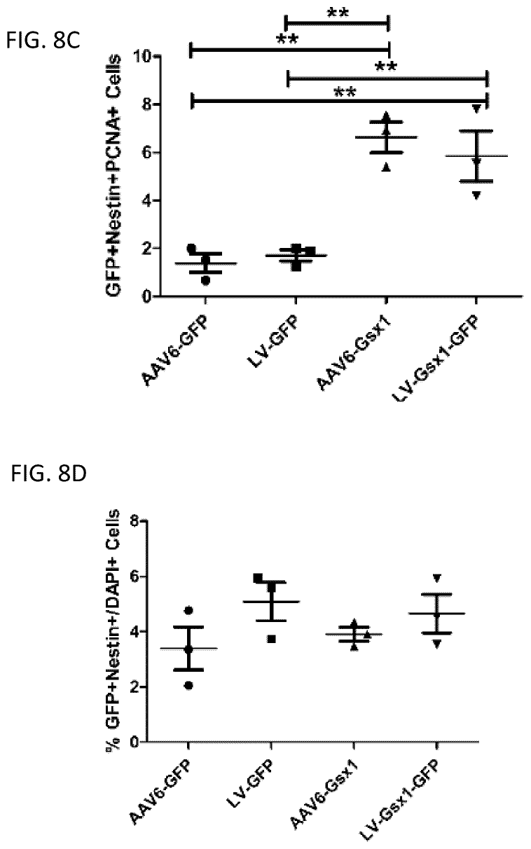

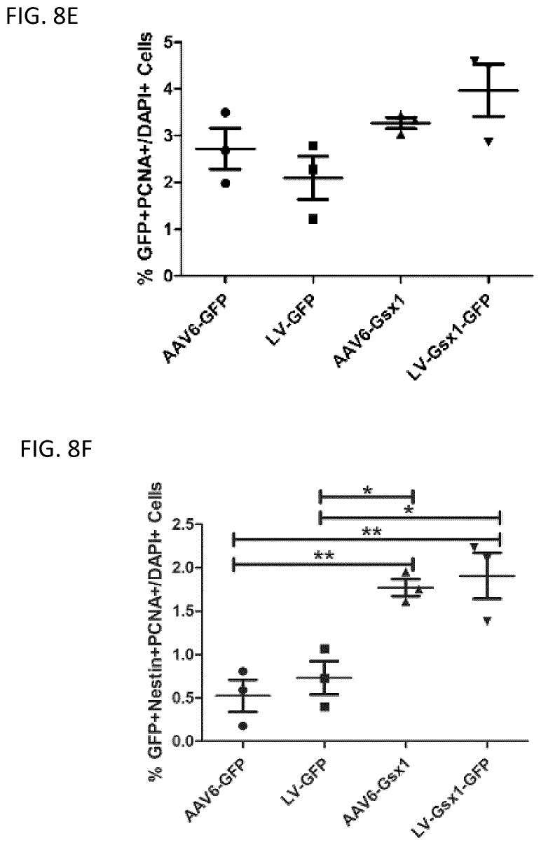

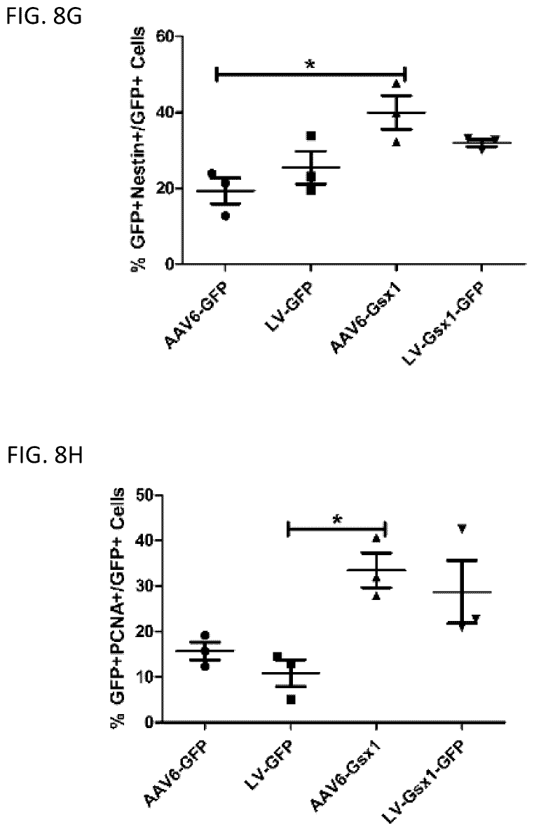

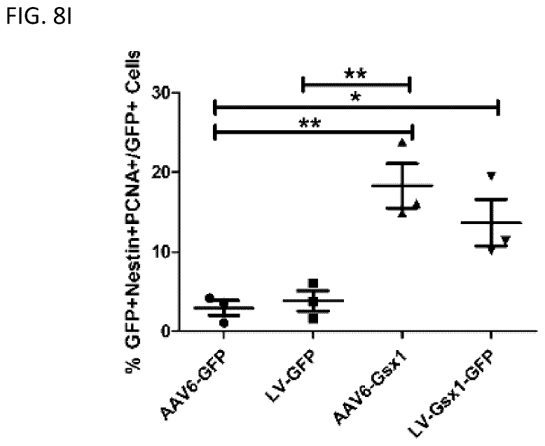

We next tested the efficacy of AAV6-Gsx1 to activate NSPCs and induce cell proliferation in the following groups: SCI+AAV6-GFP, SCI+AAV6-Gsx1, SCI+LV-GFP, SCI+LV-Gsx1-GFP in a rat model of lateral hemisection SCI. We sacrificed animals and harvested spinal cords at 4 dpi. IHC analysis was used to quantify the expression of Nestin (NSPCs) and PCNA (proliferation cell nuclear antigen). We found co-labeled Nestin+/GFP+ cells throughout and immediately adjacent to the lateral hemisection injury site and expressed this value over total GFP+ cells to represent the virus induced NSPC activation (data not shown). We also expressed this value as a percentage of total cells (DAPI+) and raw cell values (FIGS. 8A-8I)

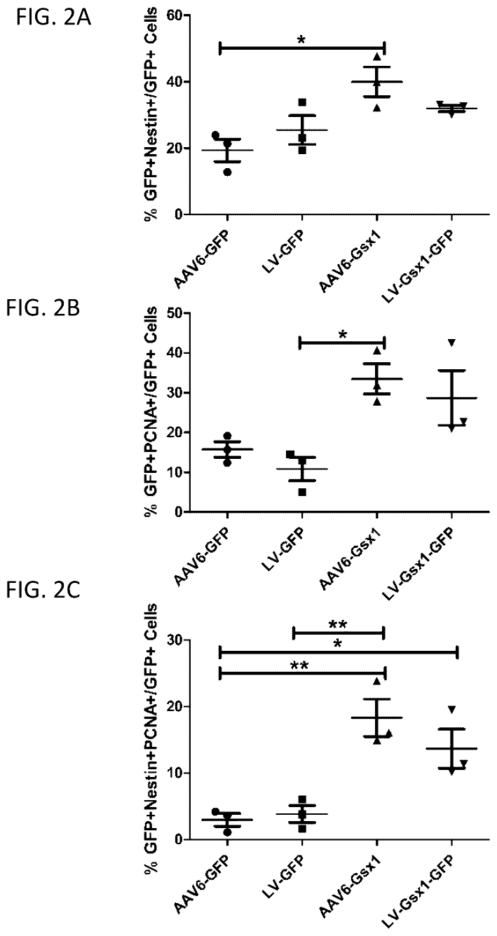

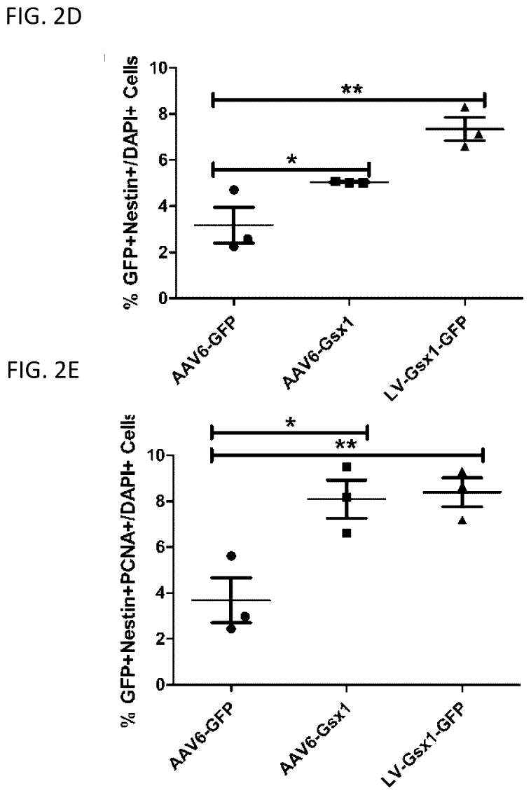

Cell count analysis showed that AAV6-Gsx1 (39.98%±4.45; n=3) and LV-Gsx1-GFP (31.96%±0.96; n=3) significantly increased Nestin+ NSPC activation in comparison with controls: LV-GFP (25.45%±4.32; n=3) and AAV6-GFP (19.36%±3.36; n=3) (FIG. 2A). We also found many co-labeled PCNA+/GFP+ cells throughout the tissue surrounding the injury and injection sites and expressed this value over GFP+ cells to quantify virus-induced proliferation. We found that AAV6-Gsx1 (33.49%±3.79; n=3) and LV-Gsx1-GFP (28.71%±6.91; n=3) significantly increased cell proliferation in comparison with controls LV-GFP (10.86%±2.94; n=3) and AAV6-GFP (15.74%±1.97; n=3) (FIG. 2B). We further investigated Gsx1-induced neurogenesis by quantifying cells with the co-labeling of markers: virally infected GFP+ proliferating PCNA+ NSPC Nestin+. We observed many Gsx1-induced co-labeled neurogenesis positive cells between the 1 mm rostral/caudal of the injection sites and throughout the injury. AAV6-Gsx1 (18.30%±2.80; n=3) and LV-Gsx1-GFP (13.66%±2.93; n=3) induce neurogenesis in comparison with controls LV-GFP (3.84%±1.28; n=3) and AAV6-GFP (2.97%±0.95; n=3) (data not shown), e.g., AAV6-Gsx1-induced activated proliferating NSPCs (FIG. 2C). We found that Gsx1 can activate NSPCs, increases proliferation, and induces neurogenesis in the acute injured spinal cord.

We proceeded to investigate AAV6-Gsx1-induced NSPC activation, proliferation, and neurogenesis in a more clinically relevant rat model of contusion SCI. Rats were subject to contusion SCI and injected with viral treatments in the following groups: SCI+AAV6-GFP, SCI+AAV6-Gsx1, SCI+LV-Gsx1-GFP. A total of 3.0 μl virus was injected into the spinal cord in four corners of the contusion injury site approximately 1 mm rostral and caudal to the epicenter immediately following SCI (FIG. 7C). The consistency of each contusion injury was confirmed visually during surgery and behaviorally following surgery with complete rear hind limb paralysis below the thoracic injury level. We sacrificed animals and harvested spinal cords at 4 dpi.

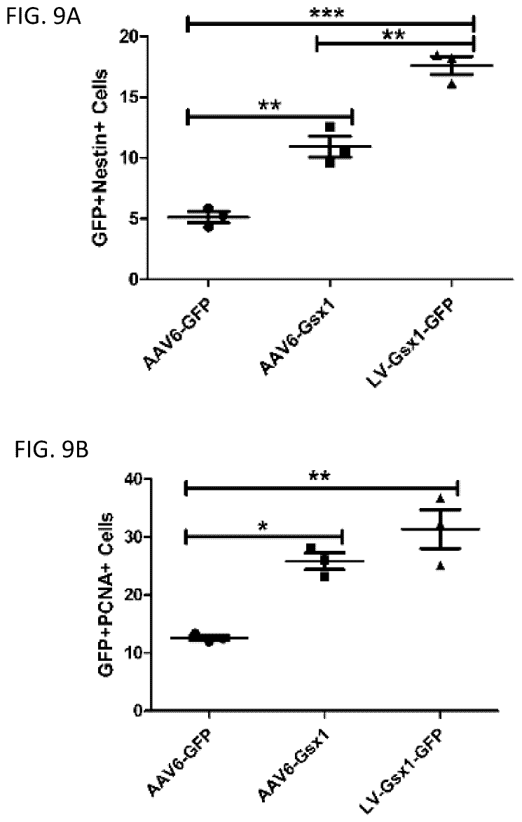

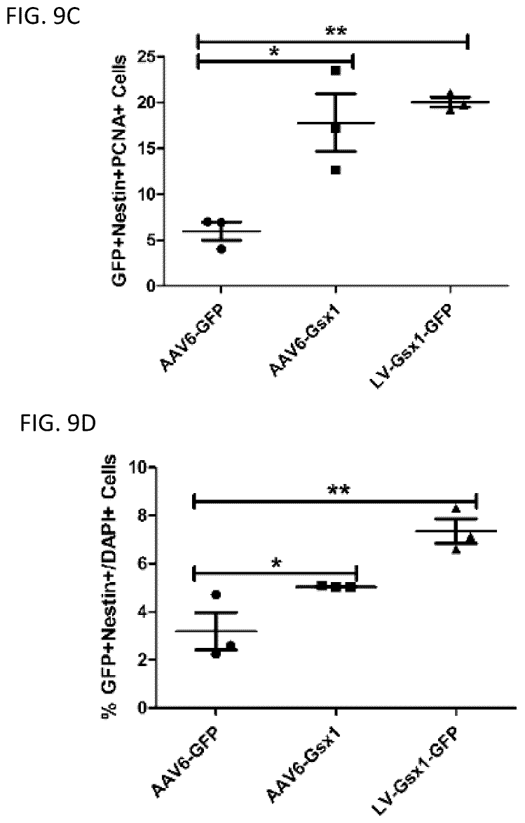

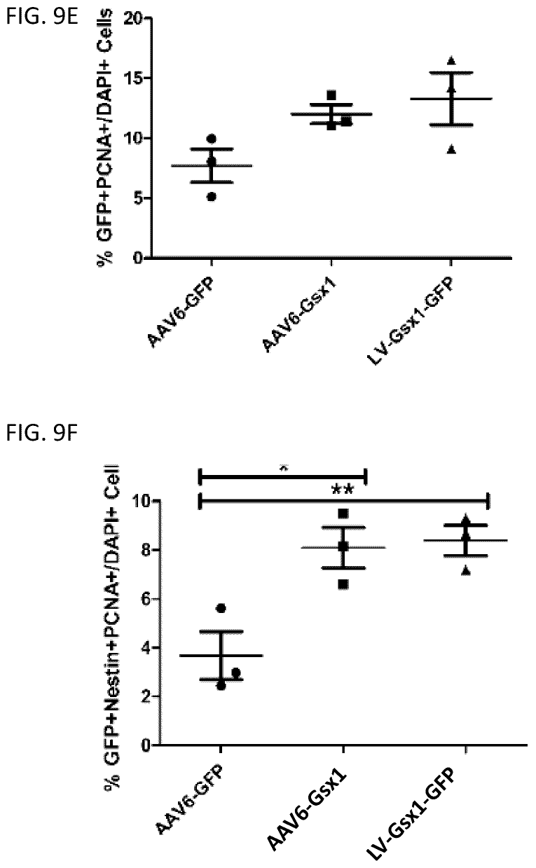

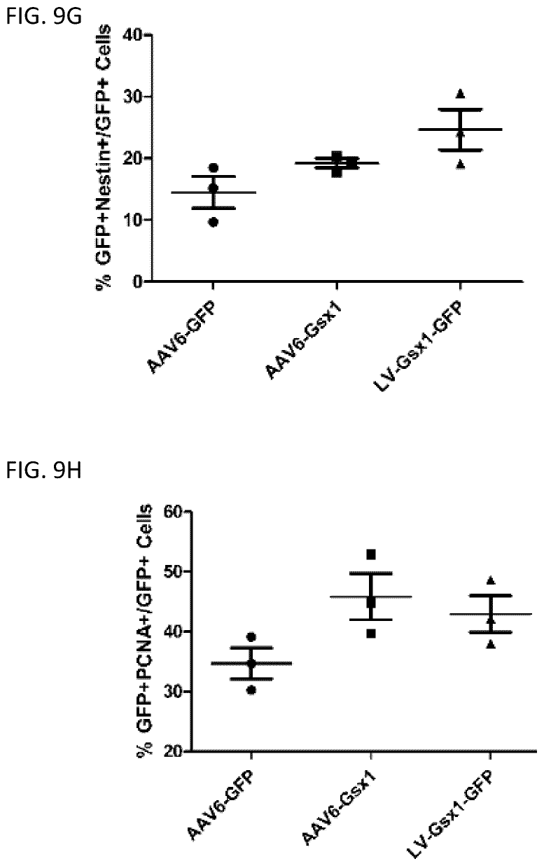

IHC analysis was used to quantify the expression of Nestin (NSPCs), Sox2 (neural progenitor cells), NG2 (glial progenitor cells/polydendrocytes), and PCNA (proliferation). The virally infected GFP+ cell signal was distributed evenly on either side of the contusion injury site, sparse in the lesion core, and consistently dispersed throughout the lesion border (data not shown). The majority of GFP+ cells were found at/near lesion or injection site and appeared to diffuse in rostral and caudal directions. The Nestin signal was prominent in the lesion border and spared rostral/caudal neural tissue. We observed many co-labeled Nestin+/GFP+ and PCNA+/GFP+ cells in the lesion border and expressed these values as a percentage of DAPI+ total cell population (data not shown) to quantify virus-induced effect on total lesion site population. We also quantified these values over total virally infected GFP+ population and raw cell values (FIGS. 9A-9I).

Cell count analysis showed that AAV6-Gsx1 (5.04%±0.02; n=3) and LV-Gsx1-GFP (7.35%±0.51; n=3) increase Nestin+ NSPC activation in comparison to control (3.18%±0.77; n=3) (FIG. 2D). The PCNA signal was less obvious but overlapped with the Nestin throughout the lesion border. We found that AAV6-Gsx1 (12.01%±0.8; n=3) and LV-Gsx1-GFP (13.29%±2.18; n=3) did not significantly increase cell proliferation in comparison with the control (7.72%±1.41; n=3), however a positive trend is obvious (FIGS. 9A-9I). To investigate neurogenesis in the NSPC populations, we observed and quantified the co-labeling of GFP+, Nestin+, and PCNA+ cells in the injured spinal cord. We found that AAV6-Gsx1 (8.09%±0.83; n=3) and LV-Gsx1-GFP (8.38%±0.63; n=3) induce neurogenesis in comparison to control (3.68%±0.98; n=3), e.g., a group of AAV6-Gsx1-induced proliferating NSPCs between the lesion core and caudal injection site (FIG. 2E).

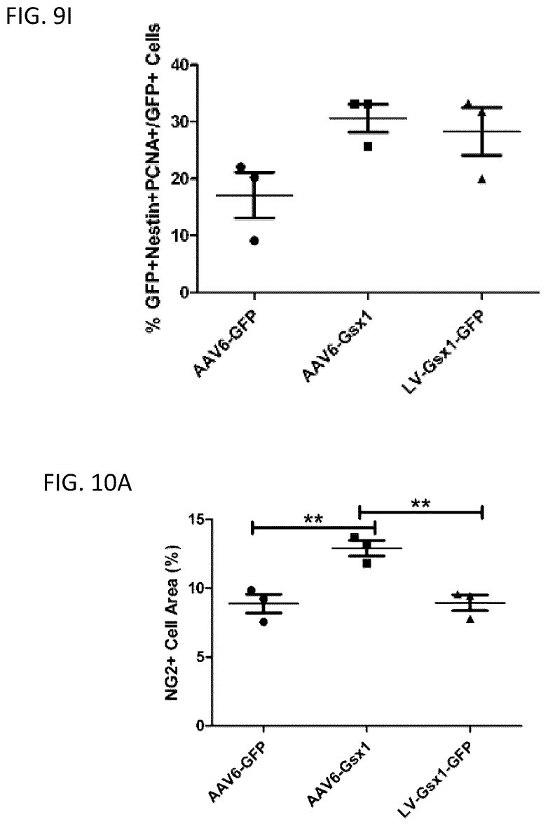

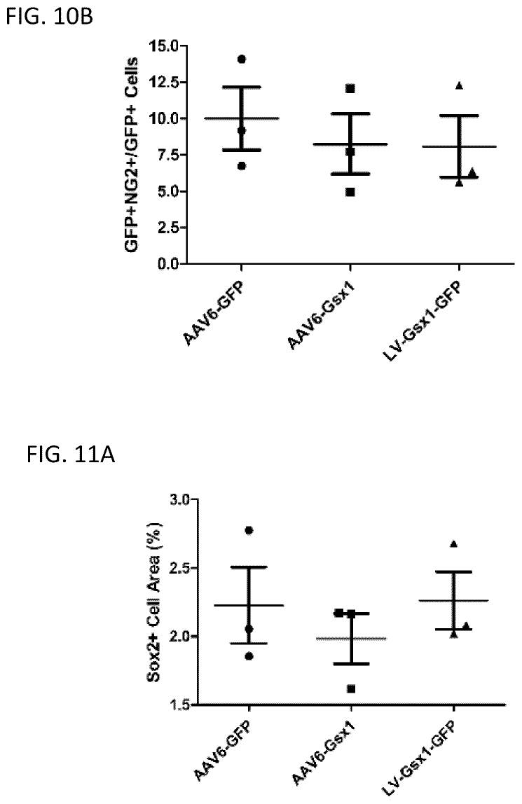

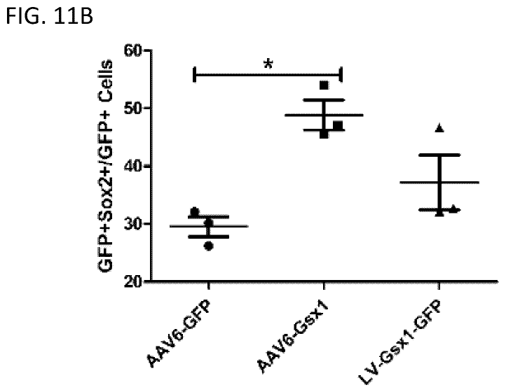

We also observed activation of NG2+ progenitors approximately 1.5 mm rostral and caudal to the injury site, counted co-labeled NG2+/GFP+ cells, and expressed over the GFP+ population (data not shown). We found that AAV6-Gsx1 (8.26%±2.07; n=3) and LV-Gsx1-GFP (8.10%±2.11; n=3) do not significantly increase NG2+ NSPC activation in comparison to the control (10.01%±2.16; n=3) (FIG. 10B). In addition, we observed Sox2 neural progenitor activation throughout the lesion site and counted co-labeled Sox2+/GFP+ cells and expressed over the GFP+ population (data not shown). We found that AAV6-Gsx1 (48.85%±2.61; n=3) significantly increased Sox2+ NSPC activation in comparison with the control (29.51%±1.74; n=3) (FIG. 11B). However, LV-Gsx1-GFP (37.21%±4.73; n=3) did not significantly activate Sox2 progenitors (FIG. 11B).

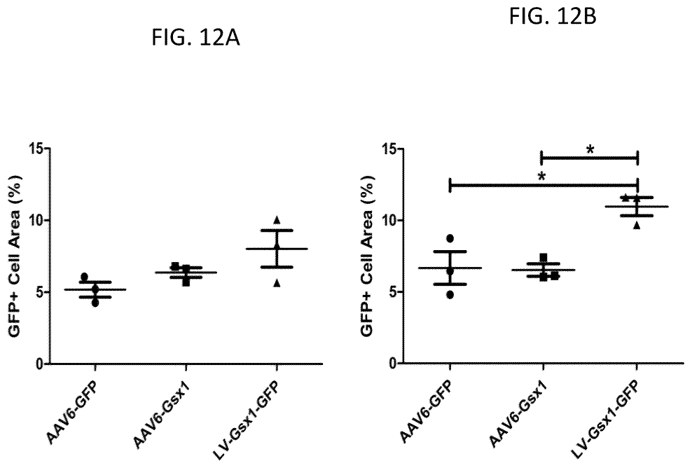

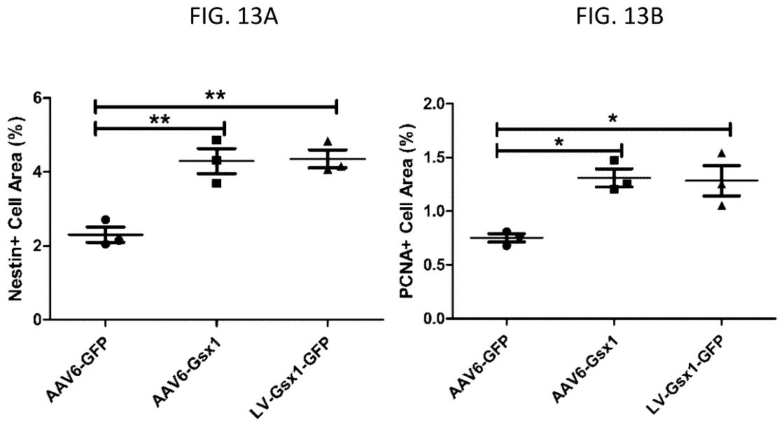

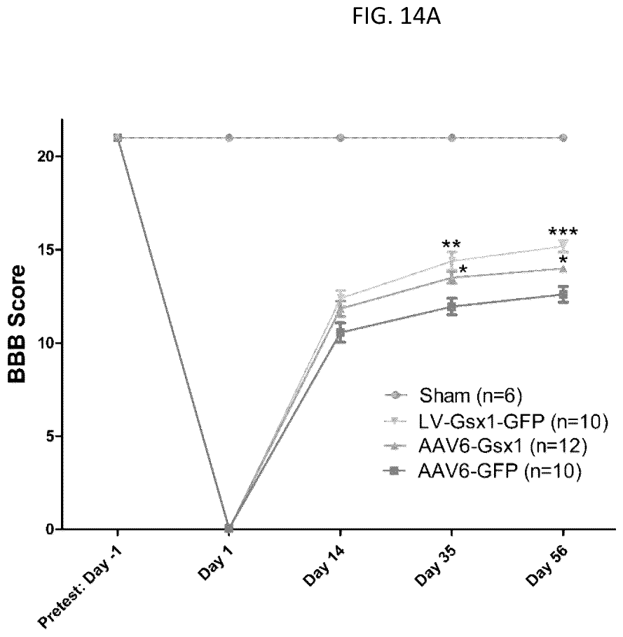

We used Ilastik, a non-biased machine learning based bioimage pixel classification analysis software to supplement our cell count analysis and found the total molecular marker signal and expressed values over total cells. We found no difference in transduction efficiency between AAV6-Gsx1 (6.37%±0.34; n=3), LV-Gsx1-GFP (8.02%±1.27; n=3), and control AAV6-GFP (5.18%±0.52; n=3) (FIG. 12A). We found that AAV6-Gsx1 (4.29%±0.34; n=3) and LV-Gsx1-GFP (4.35%±0.24; n=3) promote Nestin+ NSPC activation in comparison to control (2.30%±0.21; n=3) (FIG. 13A). We also found that AAV6-Gsx1 (1.31%±0.08; n=3) and LV-Gsx1-GFP (1.28%±0.14; n=3) increase cell proliferation in comparison with the control (0.75% %±0.04; n=3) (FIG. 13B). We investigated total NG2 progenitor activation and found that AAV6-Gsx1 (12.91%±0.57; n=3) activates NG2 polydendrocytes in comparison with the control (8.88%±0.69; n=3) (FIG. 10B). Interestingly, LV-Gsx1-GFP does not activate NG2 polydendrocytes in comparison with the control (FIG. 10A). We investigated total Sox2 progenitor activation and found that AAV6-Gsx1 (1.98%±0.18; n=3) and LV-Gsx1-GFP (2.26%±0.21; n=3) do not activate Sox2+ neural progenitors in comparison with the control (2.23%±0.28; n=3) (FIG. 11A).

Overall, Gsx1 activates various NSPC populations, increases cell proliferation, and induces neurogenesis in both the rat models of lateral hemisection and contusion SCI. The contusion SCI model is representative of the most common clinical injury and is thus used for our Gsx1 therapy efficacy analysis in three major stages: acute, subacute, and chronic.

### Example 4

### AAV6-Gsx1 Promotes Neuroblast and Immature Neuron Formation in the Subacute Contusion SCI

We next examined the presence of newborn or immature neuron formation at 14 dpi initiated by Gsx1-induced neurogenesis at 4 dpi. Rats were subject to contusion SCI and injected with viral treatments in the following three groups: SCI+AAV6-GFP, SCI+AAV6-Gsx1, SCI+LV-Gsx1-GFP. A total of 3.0 μl virus was injected into the spinal cord in four corners of the contusion injury site approximately 1 mm rostral and caudal to the epicenter immediately following SCI. Animals were sacrificed and spinal cords were harvested at 14 dpi (subacute SCI).

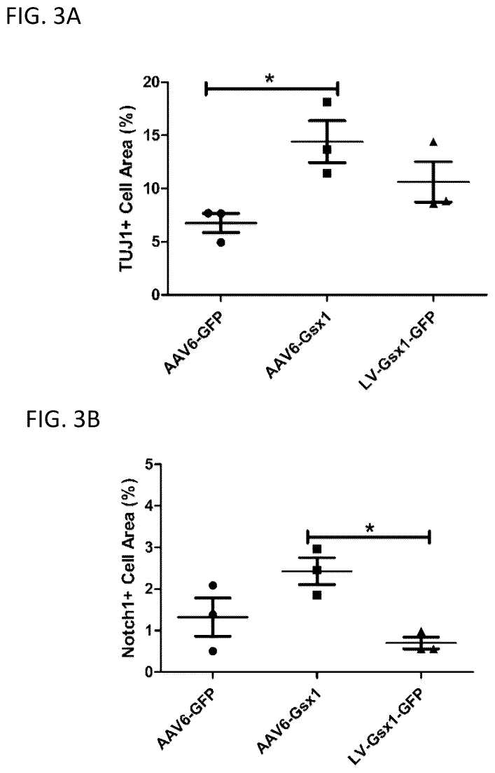

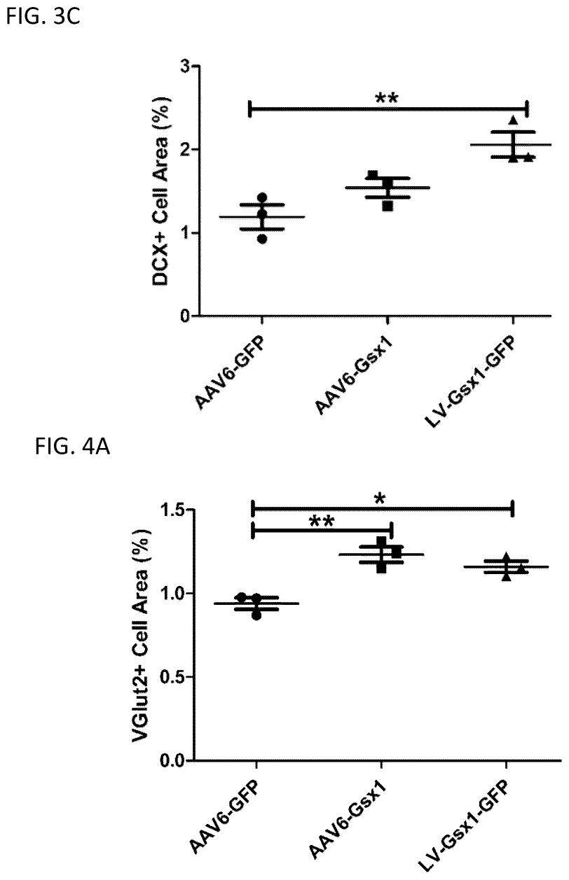

IHC analysis was used to examine the injured spinal cord for established molecular markers DCX (neuroblasts), Tuj1 (immature neurons), and Notch1 (canonical notch activity). The injured area was clear and tissue damage was extensive, spanning 1-2 mm rostral caudal to the injury epicenter (data not shown). The GFP+ cell distribution was concentrated at the injection sites and spread approximately 2 mm rostral and caudal to the lesion core. GFP+ cells were clearly present rostral and caudal to the injury epicenter, throughout the injured tissue (data not shown). We found that LV-Gsx1-GFP (10.97%±0.64; n=3) transduced a higher percentage of cells in comparison to AAV6-Gsx1 (6.53%±0.44; n=3), and control AAV6-GFP (6.67%±1.14; n=3) (FIG. 12B). Tuj1 signal was distributed throughout the injection sites and rostral/caudal to the lesion core (data not shown) We found that AAV6-Gsx1 (14.41%±1.96; n=3) significantly increased the percentage of Tuj1+ cells over total cells and LV-Gsx1-GFP (10.62%±1.89; n=3) did not in comparison to AAV6-GFP control (6.76%±0.91; n=3) (FIG. 3A). The canonical notch pathway is upregulated during cell proliferation and NSPC activation in early stages after SCI and decreases during cell differentiation. Here, we used the Notch1 marker to support Gsx1 induced differentiation, indicated by a lack of canonical pathway notch activity at 14 dpi. The Notch1 signal was evenly distributed throughout the lesion border and spared tissue 0.5 mm rostral/caudal to the injection sites (data not shown). We found that LV-Gsx1-GFP (0.70%±0.14; n=3) significantly reduced the percentage of Notch1+ cells over total cells in comparison with the AAV6-Gsx1 treatment (2.42%±0.32; n=3), supporting neuronal differentiation of LV-mediated Gsx1 activated NSPCs during subacute SCI (FIG. 3B). The DCX signal was only present at the injection sites and dissipated into the lesion core in our control SCI group (data not shown). We found that LV-Gsx1-GFP (4.73%±0.33; n=3) significantly increased the percentage of DCX+ cells over total cells, however AAV6-Gsx1 (3.36%±0.12; n=3) did not in comparison to AAV6-GFP control (2.75±0.16; n=3) (FIG. 3C).

The low percentages of newborn and immature neurons reflect the quantification area, approximately 2 mm rostral/caudal to and throughout the lesion core, and the extent of damaged tissue. Collectively, the Gsx1 gene treatments promote newborn and immature neuronal formation at 14 dpi following Gsx1-induced activation, proliferation, and neurogenesis of NSPCs at 4 dpi.

### Example 5

### AAV6-Gsx1 Increases Excitatory and Reduces Inhibitory Interneuron Populations in the Chronic Contusion SCI

The synaptic excitatory-inhibitory cell balance in the spinal cord is maintained by interneuron subtypes and required to functionally transmit signal from the brain through the spinal cord. The neurogenic gene Gsx1 drives the formation of dorsal excitatory and inhibitory interneurons during development. We demonstrated that Gsx1-induced newborn and immature neurons were generated in subacute SCI. We next investigated the role of Gsx1 on the neuronal balance and the identity of differentiated newborn and immature neurons as they develop and integrate into spinal cord neuronal circuitry. Injured animals with viral treatments were sacrificed at 56 dpi.

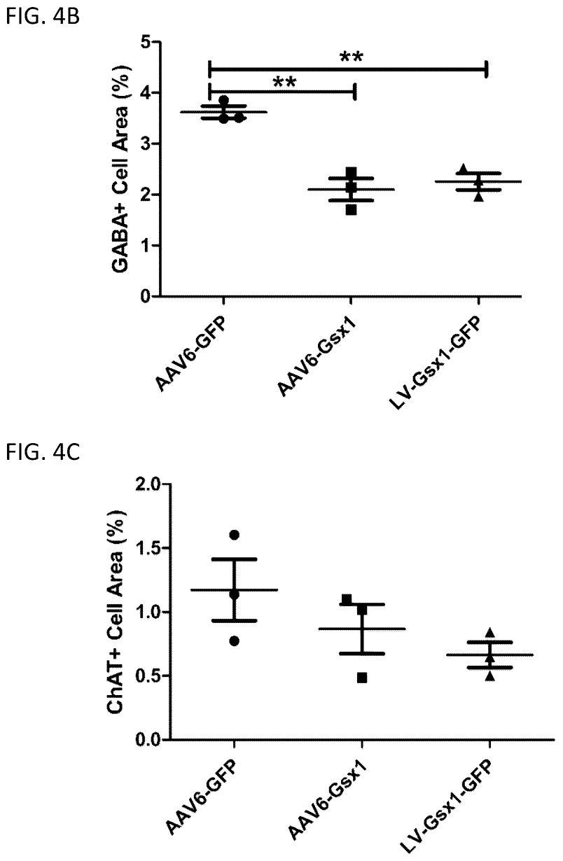

IHC analysis was used to determine the number of excitatory (VGlut2), inhibitory (GABA), and cholinergic (ChAT) interneurons. The injured area was clear and spanned 2 mm rostral/caudal to the injury epicenter. The GFP+ cell distribution was concentrated at the injection sites and spread approximately 1-2 mm rostral/caudal to the lesion core and some GFP+ cells could be found even further, indicating extensive viral spread. GFP+ cells were clearly present rostral and caudal to the injury epicenter, throughout the injured tissue (data not shown). However, no GFP+ cells were present in the injury epicenter, consistent with our findings at 4 dpi and 14 dpi. At the injury epicenter, the microenvironment is not favorable for cell growth, thus cells do not usually survive (data not shown). The vGlut2 signal was distributed throughout our control treatment only rostral and slightly caudal to the injured area. Interestingly, our treatments contained many co-labeled GFP+vGlut2+ cells throughout the lesion site spanning 4 mm rostral to caudal, indicated by yellow signal (data not shown). Treatments of AAV6-Gsx1 (1.23%±0.05; n=3) and LV-Gsx1-GFP (1.16%±0.03; n=3) increased the percentage of VGlut2+/total cells in comparison to control AAV6-GFP (0.94%±0.04; n=3) (FIG. 4A). The most prominent GABA signal was present in our control and consistent rostral and caudal to the lesion core, but not present in the lesion core. We found very few if any co-labeled GFP+GABA+ cells (data not shown). AAV6-Gsx1 (2.1%±0.22; n=3) and LV-Gsx1-GFP (2.25%±0.16; n=3) reduced the percentage of GABA+ cells among total cells in comparison to control (3.62%±0.12; n=3) (FIG. 4B). The ChAT signal was distributed evenly throughout the rostral spinal cord but interrupted by the lesion site and not present caudal to the lesion. Notably, AAV6-Gsx1 (0.87%±0.19; n=3) and LV-Gsx1-GFP (0.66%±0.10; n=3) did not increase ChAT+ cells in comparison to AAV6-GFP control (1.17%±0.24; n=3) (FIG. 4C).

Overall, Gsx1 alters the excitatory-inhibitory cell balance in the chronic injured spinal cord by reducing inhibition and increasing excitation at the lesion core. The large number of co-labeled virally infected excitatory interneurons in our AAV6-Gsx1 and LV-Gsx1-GFP treatments may indicate that the newborn and immature neurons formed at 14 dpi have differentiated into excitatory interneurons at 56 dpi.

### Example 6

### AAV6-Gsx1 Reduces Reactive Gliosis and Glial Scar Formation in the Subacute and Chronic SCI

The glial scar presents a physical and chemical barrier to regeneration due to a dense astrocyte/fibroblast cell layer, thick secreted ECM, and inhibitory molecules, e.g., CSPGs, collogen. NSPCs play a significant role in scar border formation and contribute glial fate progeny to the astrocyte scar populations. Gsx1 promotes newborn and immature neuronal populations in subacute SCI. We also identified that these populations differentiate into excitatory and not inhibitory interneurons.

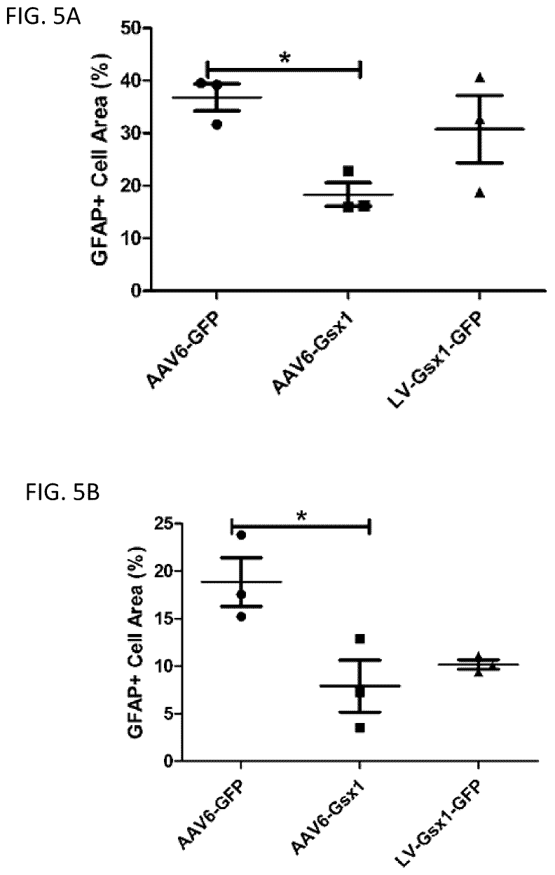

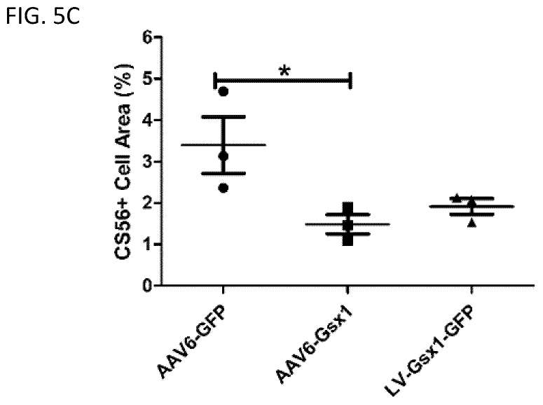

We next investigated the effect of Gsx1 on reactive gliosis and glial scar formation at 14 dpi and 56 dpi. IHC analysis was used to determine the expression of GFAP (reactive astrocytes) at 14 dpi and CS56 (CSPGs) and GFAP (astrocyte density) in the mature glial scar at 56 dpi. The GFAP signal distribution at 14 dpi was most prominent in the spared neural tissue adjacent to the lesion site, and clearly astrocytes were elongating processes to begin formation of the glial scar (data not shown). We found that AAV6-Gsx1 (18.32%±2.22; n=3) reduced reactive gliosis (GFAP/total cells) in comparison to AAV6-GFP control (36.79%±2.56; n=3) at 14 dpi (FIG. 5A). The CS56 signal distribution at 56 dpi was diffuse and most densely occurring at the scar border at the edge of the lesion core but spread 2 mm rostral/caudal to the injury site (data not shown). AAV6-Gsx1 (1.48%±0.23; n=3) reduced CSPG deposition (CS56/total cells) in comparison to AAV6-GFP control (3.40%±0.69; n=3) at 56 dpi (FIG. 5B). The GFAP distribution formed a clear dense border surrounding the injury site with diffuse signal spreading 0.5-1 mm away from the injury scar border (data not shown). AAV6-Gsx1 (7.91%±2.73; n=3) also reduced glial scar border astrocyte density (GFAP/total cells) in comparison to AAV6-GFP control (18.86%±2.56; n=3) at 56 dpi (FIG. 5C). Interestingly, LV-Gsx1-GFP did not significantly reduce reactive gliosis (27.77%±3.53; n=3) at 14 dpi (FIG. 5A), CSPGs (1.91%±0.19; n=3) (FIG. 5B) and astrocyte density (10.18%±0.49; n=3) (FIG. 5C) at 56 dpi compared with the AAV6-GFP control, however displayed a trend toward glial scar reduction. These results indicate that AAV6-Gsx1 reduced astrocyte populations during reactive gliosis and scar border maturation. Thus, our Gsx1-transduced NSPCs produced less glial fated cells, e.g., astrocyte subtypes, and instead promoted differentiation into neuronal subtypes such as excitatory interneurons.

### Example 7

### Gsx1 Promotes 5-HT Neuronal Activity and Locomotor Functional Recovery in the Chronic SCI

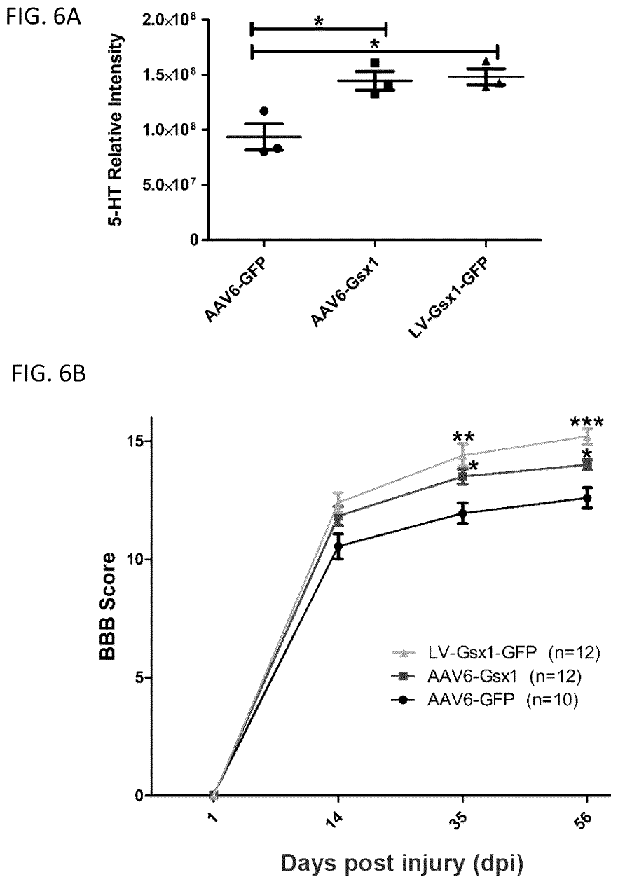

The serotonergic (5-HT) neuronal activity is required for the normal transmission of signal in the spinal cord to generate autonomic, motor, and sensory function. Locomotor function is directly impacted by 5-HT activity, by modulating spinal network activity required for motor control. After SCI, a loss of serotonergic (5-HT) projections occurs resulting in innervation of motoneurons. Thus, the restoration of 5-HT neuronal activity is necessary to promote effective signal transmission through motor circuits in the injured spinal cord and facilitate locomotor recovery. To examine this, we performed IHC to examine 5-HT neuronal activity at 56 dpi. The 5-HT signal was extremely dense and distributed in parallel projections from rostral to caudal. The rostral signal was interrupted by the lesion core and did not continue into caudal spinal cord in our control (data not shown). We found that AAV6-Gsx1 (6.54%±0.46; n=3) and LV-Gsx1-GFP (6.56%±0.30; n=3) increased 5-HT immuno-reactivity (5HT/total cells) in comparison to AAV6-GFP control (4.27%±0.56 n=3) at 56 dpi (FIG. 6A). In our treatments, 5-HT neuronal activity continues through the lesion core in two ways: (1) directly through the lesion core with no interruption in the AAV6-Gsx1 group, (2) around the injury epicenter and penetrating through the scar border in the LV-Gsx1-GFP group. Thus, Gsx1 promotes restoration of neuronal activity and sprouts neuronal circuits through the lesion core in chronic SCI.

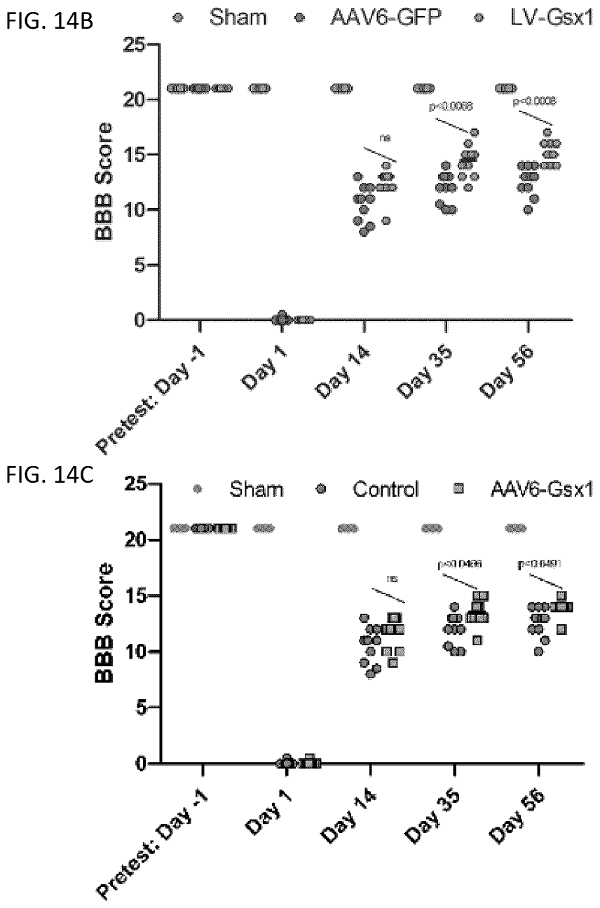

To examine the effect of Gsx1 therapy on the locomotor functional recovery in injured animals, a blinded analysis of an open field locomotor test was performed with the Basso, Beattie, Bresnahan (BBB) locomotor scoring scale assessed at 1, 14, 35, and 56 dpi. BBB scores in rats with the injection of AAV6-Gsx1 (13.5±0.31 at 35 dpi and 14±0.21 at 56 dpi; n=12) and LV-Gsx1-GFP (14.4±0.48 at 35 dpi and 15.2±0.33 at 56 dpi; n=10) show significant increase accompanied with functional locomotor recovery compared with the AAV-GFP control (11.95±0.44 at 35 dpi and 12.6±0.43 at 56 dpi; n=10) (FIG. 6B and FIGS. 14A-14C).

The BBB locomotor scoring scale is divided into three major stages of recovery: early (1-7), intermediate (8-13), and late stage (14-21). At 35 dpi, differences between control AAV6-GFP and treated animals indicate that Gsx1 rescued coordination of injured animals. Our controls (SCI+AAV6-GFP) remained in the intermediate stage, defined by uncoordinated and inconsistent hind limp plantar stepping, and our treatment groups ascended into the late stage, defined by coordination of front and hind limbs and consistent plantar stepping. At 56 dpi, differences between control AAV6-GFP and treatments AAV6-Gsx1 or LV-Gsx1-GFP show that Gsx1 treated animals continued to improve coordination between front and hind limbs and display consistent plantar stepping, whereas control animals still showed uncoordinated movement and inconsistent plantar stepping in the hind limbs. Overall, the Gsx1 therapy resulted in the restoration of coordinated function in the hind limbs, consistent weight bearing plantar stepping beginning at 5 weeks, and development of variable coordination between forelimbs and hindlimbs at 8 weeks. In contrast, complete hind limb coordination was never observed in the control animals. However, Gsx1 therapy had no effect on restoring bladder function compared to the control AAV6-GFP.

### Example 8

Gsx1 does not Change Endogenous Neuron Function after SCI

Neuronal degeneration, demyelination, dysfunction and death occur after SCI due to primary mechanical damage and prolonged inflammatory response in the acute and subacute SCI phases. To rule out any secondary effects of the Gsx1 therapy and account for the established AAV6 neuronal tropism, we investigated Gsx1-mediated changes in neuron populations at 14 dpi. IHC analysis was used to examine the injured spinal cord for established molecular markers MAP2 or NeuN (mature neurons), Caspase-3 (cell death), 5-HT (serotonergic neuronal activity), Myelin Basic Protein (myelination), and Synaptophysin (synapses).

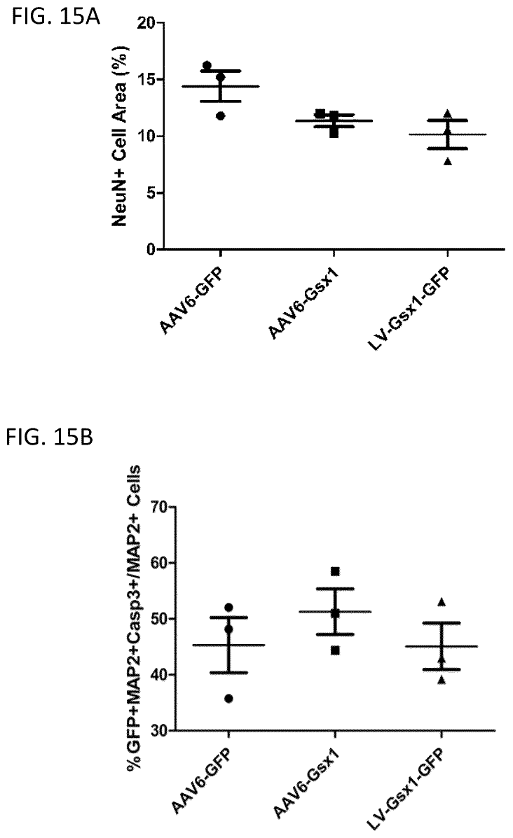

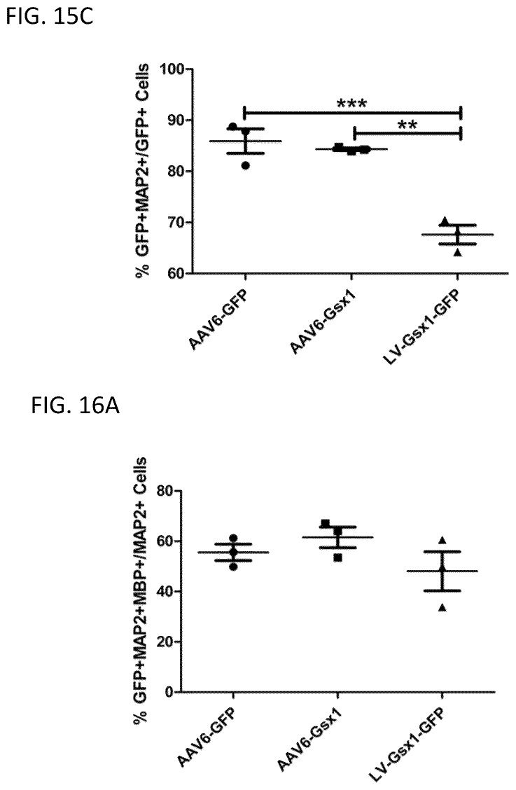

Fluorescence imaging of mature neurons was conducted approximately 2 mm away from the lesion core due to high neuronal cell death. The NeuN and MAP2 mature neuron signal was observed 2 mm rostral to the lesion core and not present caudal (data not shown). We found no significant difference between LV-Gsx1-GFP (10.14%±1.24; n=3) and AAV6-Gsx1 (11.36%±0.54; n=3) in percentage of NeuN+ cells over total cells and the AAV6-GFP control (14.41%±1.34; n=3) (FIG. 15A). Cell counting analysis shows the percentage of GFP+ and MAP2+ cells among virally infected GFP+ cells in AAV6-Gsx1 (84.33%±0.25; n=3) and AAV-GFP (85.91%±2.39; n=3) were significantly greater than LV-Gsx1-GFP (67.63%±1.83; n=3) treatment (supplemental FIG. 15C). The Caspase-3 (Casp-3) signal was concentrated around the lesion core and dispersed 1 mm rostral/caudal (data not shown). We found that AAV6-Gsx1 (51.29%±4.08; n=3) and LV-Gsx1-GFP (45.09%±4.15; n=3) do not enhance neuron survival (GFP+/Casp-3+/MAP2+ cells) in comparison to AAV-GFP (45.32%±4.92; n=3) control (FIG. 15B).

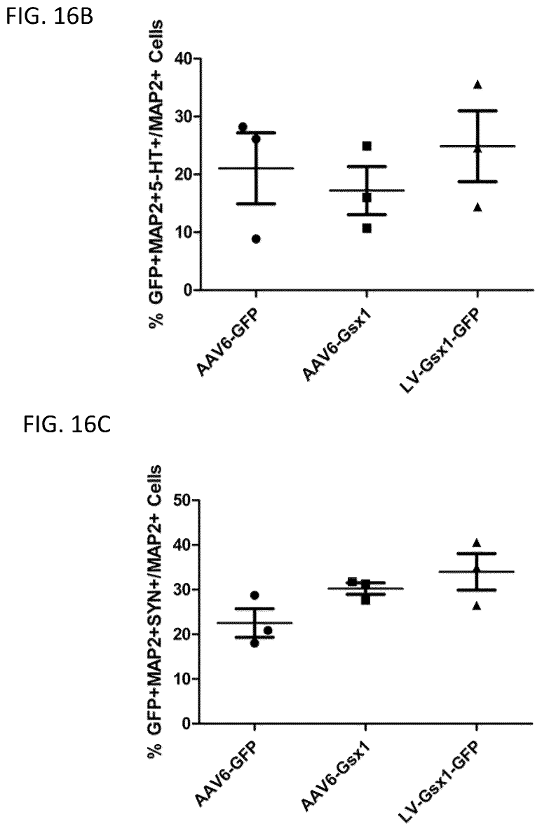

The Myelin Basic Protein (MBP) signal was observed rostral to and throughout the lesion core border (data not shown). We found that AAV6-Gsx1 (61.54%±4.12; n=3) and LV-Gsx1-GFP (48.09%±7.77; n=3) do not increase neuron myelination (GFP+/MBP+/MAP2+ cells) in comparison to AAV-GFP (55.58%±3.28; n=3) control (FIG. 16A). The 5-HT signal was observed clearly rostral to the lesion core and was not present caudal (data not shown). We found that AAV6-Gsx1 (17.12%±4.16; n=3) and LV-Gsx1-GFP (24.87%±6.12; n=3) do not promote serotonergic neuronal activity (GFP+/5-HT+/MAP2+ cells) in comparison to AAV-GFP (21.06%±6.14; n=3) control (FIG. 16B). The Synaptophysin signal was distributed rostral and caudal to lesion core (data not shown). We also found that AAV6-Gsx1 (30.21%±1.29; n=3) and LV-Gsx1-GFP (33.98%±4.09; n=3) do not promote neuronal synaptogenesis (GFP+/SYN+/MAP2+ cells) in comparison to AAV-GFP (22.52%±3.20; n=3) control (FIG. 16C).

Overall, the Gsx1 treatments infect mature neurons but do not enhance neuronal survival, serotonergic neuronal activity, myelination, or synapse formation at 14 dpi. This suggests that Gsx1-induced functional locomotor recovery is due to neurogenesis at 4 dpi, newborn neuron formation at 14 dpi, and regeneration of neurons and neuronal activity at 56 dpi.

It will be apparent that the precise details of the methods or compositions described may be varied or modified without departing from the spirit of the described aspects of the disclosure. We claim all such modifications and variations that fall within the scope and spirit of the claims below.

We claim:

1. A method of treating a neurological disorder in a mammalian subject, comprising administering to the subject a therapeutically effective amount of an adeno-associated virus 6 (AAV6) vector or virus comprising a heterologous nucleic acid molecule.

2. The method of claim 1, wherein the heterologous nucleic acid encodes Gsx1.

3. The method of claim 2, wherein the heterologous nucleic acid encodes a Gsx1 protein with at least 90%, at least 95%, at least 96%, at least 97%, at least 98%, at least 99% or 100% sequence identity to the amino acid sequence of SEQ ID NO: 3, SEQ ID NO: 6, or SEQ ID NO: 10.

4. The method of claim 3, wherein the heterologous nucleic acid encoding Gsx1 comprises a nucleic acid with at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9.

5. The method of claim 1, wherein the heterologous nucleic acid is operably linked to a promoter.

6. The method of claim 5, wherein the promoter is a constitutive promoter or a central nervous system (CNS)-specific promoter.

7. The method of claim 6, wherein the constitutive promoter is a CMV promoter.

8. The method of claim 1, wherein the neurological disorder is a spinal cord injury, a brain injury, or both.

9. The method of claim 8, wherein the spinal cord injury, brain injury, or both is caused by a vehicle crash, fall, act of violence, sports, or other physical trauma.

10. The method of claim 1, wherein the neurological disorder is Parkinson's disease, Alzheimer's disease, stroke, ischemia, epilepsy, Huntington's disease, multiple sclerosis, or amyotrophic lateral sclerosis.

11. The method of claim 1, wherein the administering comprises injection.

12. The method of claim 11, wherein the injection comprises injection into the CNS.

13. The method of claim 1, wherein the subject is a human subject.

14. The method of claim 1, wherein the therapeutically effective amount of the AAV6 vector or virus comprising a heterologous nucleic acid molecule is present in a pharmaceutical composition.

15. The method of claim 1, wherein the administering occurs within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the neurological disorder.

16. A composition comprising an adeno-associated virus 6 (AAV6) vector or virus comprising a nucleic acid molecule encoding a Gsx1 protein.

17. The composition of claim 16, wherein the heterologous nucleic acid encodes a Gsx1 protein with at least 90%, at least 95%, at least 96%, at least 97%, at least 98%, at least 99% or 100% sequence identity to the amino acid sequence of SEQ ID NO: 3, SEQ ID NO: 6, or SEQ ID NO: 10.

18. The composition of claim 17, wherein the heterologous nucleic acid encoding Gsx1 comprises a nucleic acid with at least 90%, at least 95%, at least 96%, at least 97%, at least 98%, at least 99% or 100% sequence identity to SEQ ID NO: 1, SEQ ID NO: 2, SEQ ID NO: 4, SEQ ID NO: 5, SEQ ID NO: 8, or SEQ ID NO: 9.

19. A method of expressing a heterologous nucleic acid in neural stem and progenitor cells (NSPCs), comprising transducing NPSCs with an adeno-associated virus 6 (AAV6) vector or virus comprising the heterologous nucleic acid.

20. The method of claim 19, wherein the NPSCs are spinal cord NPSCs and/or wherein the NPSCs are in an injured or disease state.


SEQUENCE LISTING (9 sequences)

SEQ ID NO: 1 - DNA, 1477 bp, Mus musculus
  source 1..1477: mol_type = other DNA
cagcagcagccaaggtgattccagcccgggcttgagccgcgagtggagcctccggggcccgggaagctgcgggtggccgcggccaggggaagctacgacaggatctgcagttccctcgggctccaggggcgggctggcggcaggtggacctcgcgctggagccatgccgcgctccttcctggtggattcccttgtgctgcgggaagccagcgacaagaaggctccggagggcagcccgccaccgctcttcccctacgcggtcccgccgccgcacgcgctccacggcctctcgccgggcgcctgccacgcgcgcaaggccggcttgctgtgcgtgtgtcccctctgtgtcaccgcttcgcagctgcacgggccccccgggccgccggcactgccgctactcaaggcgtccttccctcccttcggatcgcagtactgccacgcacccctgggccgccagcactccgtgtcccctggagtcgcccacggcccggccgcggccgcagcagcagctgcactctaccagacctcctacccgctgccggatcccagacagtttcactgcatctctgtggacagcagctcgaaccagctgcccagcagcaagaggatgcggacggcgttcaccagcacacagctcctggagctggagcgagagttcgcctccaacatgtacctctcccgcctgcggcgcatcgagatcgcgacctatctgaacctgtccgagaagcaggtgaagatctggtttcagaaccgccgggtgaagcacaagaaagaaggcaaaggcagtaaccaccgcggcggagctggggcgggggccggcgggggcgcaccgcaaggctgcaagtgctcttcgctctcctcagccaaatgctcagaggacgacgacgaattgcccatgtctccatcttcctccgggaaggatgacagagatctcacagtcactccgtaggtgcgccttttagaggaccattggtttccccaccccccaccccgacccttcccgcacttcaaagactggtccccaggcacccgctggccaaccgacggatttcgttgggctttgcggtggtgcgcagctctaggcaaagctaagaccttagcagacacttgaagacagtgcccctgtcccttgggcttcagggtgtttaggaggactccaagcgatgaaggctgagtcctcctcctaggacacagcctcttctcccaggcacgcaggccggagcacagcgccttgctgaccgccagcgcctcttcgcctgccaactctgggctggttcaagcttcctcggttccactagtctctccttctcggtcaatctgggcttttcactccgcgagtggctgtttgcttttcttttaacatttctttcttgcccccaactcgtctccccccactgtggtcctttatgcaacacgtctatggacttaacttttccttccctcctcaggaagtctctcccttctgtccgtttgtccc

SEQ ID NO: 2 - DNA, 786 bp, Mus musculus
  source 1..786: mol_type = other DNA
atgccgcgctccttcctggtggattcccttgtgctgcgggaagccagcgacaagaaggctccggagggcagcccgccaccgctcttcccctacgcggtcccgccgccgcacgcgctccacggcctctcgccgggcgcctgccacgcgcgcaaggccggcttgctgtgcgtgtgtcccctctgtgtcaccgcttcgcagctgcacgggccccccgggccgccggcactgccgctactcaaggcgtccttccctcccttcggatcgcagtactgccacgcacccctgggccgccagcactccgtgtcccctggagtcgcccacggcccggccgcggccgcagcagcagctgcactctaccagacctcctacccgctgccggatcccagacagtttcactgcatctctgtggacagcagctcgaaccagctgcccagcagcaagaggatgcggacggcgttcaccagcacacagctcctggagctggagcgagagttcgcctccaacatgtacctctcccgcctgcggcgcatcgagatcgcgacctatctgaacctgtccgagaagcaggtgaagatctggtttcagaaccgccgggtgaagcacaagaaagaaggcaaaggcagtaaccaccgcggcggagctggggcgggggccggcgggggcgcaccgcaaggctgcaagtgctcttcgctctcctcagccaaatgctcagaggacgacgacgaattgcccatgtctccatcttcctccgggaaggatgacagagatctcacagtcactccgtag

SEQ ID NO: 3 - protein, 261 aa, Mus musculus
MPRSFLVDSLVLREASDKKAPEGSPPPLFPYAVPPPHALHGLSPGACHARKAGLLCVCPLCVTASQLHGPPGPPALPLLKASFPPFGSQYCHAPLGRQHSVSPGVAHGPAAAAAAAALYQTSYPLPDPRQFHCISVDSSSNQLPSSKRMRTAFTSTQLLELEREFASNMYLSRLRRIEIATYLNLSEKQVKIWFQNRRVKHKKEGKGSNHRGGAGAGAGGGAPQGCKCSSLSSAKCSEDDDELPMSPSSSGKDDRDLTVTP

SEQ ID NO: 4 - DNA, 1823 bp, Homo sapiens
  source 1..1823: mol_type = other DNA
accactagcgctggccagcaccccgcgctctttgggcggtgcccacggcagcagaggctactgtttcagcctaggtctcagccgcgcgttcagcctcctgggcagaggcagctgcgggataccgcggccagggaaagcgcgtggagagccgaaaggtgcggtgggcgcagagggcgggctggctgcggggcgaccgcgcgccggggccatgccgcgctccttcctggtggactcgctagtgctgcgcgaggcgggcgagaagaaggcgcccgagggcagcccgccgccgctcttcccctacgctgtgcccccgccgcacgcgctgcacggtctctcgcctggcgcctgccacgcgcgcaaggctgggctgctgtgcgtgtgcccgctctgcgtcaccgcctcgcagctgcatgggccccccgggccgcccgcgctgcctctactcaaggcttccttcccacccttcggctcgcagtactgccacgcgcccctgggccgccagcactctgctgtgtcgcccggggtcgctcacggcccggccgccgctgctgctgccgccgcgctctaccagacctcctacccgctgcctgaccccaggcagttccactgcatctctgtggacagcagctctaaccagctgcccagcagcaagaggatgcgcacggctttcaccagcacgcagctgctagagctggagcgcgagttcgcttctaatatgtacctgtcccgcctacgtcgcatcgagatcgcgacctacctgaatctgtccgagaagcaggtgaagatctggtttcagaaccgccgagtgaagcacaagaaggagggcaagggcagcaaccatcgtggcggcggcggcgggggtgccggtggtggcgggagcgcaccgcaaggctgcaagtgcgcatcgctctcctcagccaagtgctccgaggatgacgacgaattgcccatgtctccgtcctcctcagggaaggacgaccgggatcttacggtcactccctaggcgcgtgtctccctaggtcgcccaccccaagacctccctgcgcctcggagactagtcctgggactcagcgctgattcccaggcacccgcagccaaaccactgcctggcatggatttggcactgctttgcagaggtcccgggcctgggcagggctgagagcttggcagagactggaccatggcgtccccgccccagggctcgctcgtgtccaaagccaactccaagctgtgaacactgtaagcgctcgagtcctcctgggcagtgcagcaccgcggcccccgcccgcaagcctgcttggcgcagcgccttgctggcagccggcgcctcctagcccgccctctctgggctggcttgggcttcctcgcttactctactctcctccgctctcggtcaaggtcggcggccttggatgctcgttcgcttctctttttaaaatgtctttttgtccctccccacctctttcctgtgatcatttattcactcggcccccgcccgccaacacacacatttatgaacccccactttttttttccttctccttttcccttcccctcctcagggagcctcctccttcagtcagtccatttgtcccttgatctggccttgctgtcctcagtccccacggctcctctcacagatgataaatttcgcccgtagtatccatattggggaaacagatttgctctgtttccaacacgctcttccttcctctaaacataggaccccgctctgcgccgctggcgaccccactcttccccctccagactctggcccgccccagcctagctgtgtaattgtacggcctctgcaatgccagaag

SEQ ID NO: 5 - DNA, 795 bp, Homo sapiens
  source 1..795: mol_type = other DNA
atgccgcgctccttcctggtggactcgctagtgctgcgcgaggcgggcgagaagaaggcgcccgagggcagcccgccgccgctcttcccctacgctgtgcccccgccgcacgcgctgcacggtctctcgcctggcgcctgccacgcgcgcaaggctgggctgctgtgcgtgtgcccgctctgcgtcaccgcctcgcagctgcatgggccccccgggccgcccgcgctgcctctactcaaggcttccttcccacccttcggctcgcagtactgccacgcgcccctgggccgccagcactctgctgtgtcgcccggggtcgctcacggcccggccgccgctgctgctgccgccgcgctctaccagacctcctacccgctgcctgaccccaggcagttccactgcatctctgtggacagcagctctaaccagctgcccagcagcaagaggatgcgcacggctttcaccagcacgcagctgctagagctggagcgcgagttcgcttctaatatgtacctgtcccgcctacgtcgcatcgagatcgcgacctacctgaatctgtccgagaagcaggtgaagatctggtttcagaaccgccgagtgaagcacaagaaggagggcaagggcagcaaccatcgtggcggcggcggcgggggtgccggtggtggcgggagcgcaccgcaaggctgcaagtgcgcatcgctctcctcagccaagtgctccgaggatgacgacgaattgcccatgtctccgtcctcctcagggaaggacgaccgggatcttacggtcactccctag

SEQ ID NO: 6 - DNA, 6751 bp, synthetic construct
  source 1..6751: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaaggcaattgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattgtcgacaagctagccaaagcttcaggtaccagggatccacgaattcgcccttcagcagcagccaaggtgattccagcccgggcttgagccgcgagtggagcctccggggcccgggaagctgcgggtggccgcggccaggggaagctacgacaggatctgcagttccctcgggctccaggggcgggctggcggcaggtggacctcgcgctggagccatgccgcgctccttcctggtggattcccttgtgctgcgggaagccagcgacaagaaggctccggagggcagcccgccaccgctcttcccctacgcggtcccgccgccgcacgcgctccacggcctctcgccgggcgcctgccacgcgcgcaaggccggcttgctgtgcgtgtgtcccctctgtgtcaccgcttcgcagctgcacgggccccccgggccgccggcactgccgctactcaaggcgtccttccctcccttcggatcgcagtactgccacgcacccctgggccgccagcactccgtgtcccctggagtcgcccacggcccggccgcggccgcagcagcagctgcactctaccagacctcctacccgctgccggatcccagacagtttcactgcatctctgtggacagcagctcgaaccagctgcccagcagcaagaggatgcggacggcgttcaccagcacacagctcctggagctggagcgagagttcgcctccaacatgtacctctcccgcctgcggcgcatcgagatcgcgacctatctgaacctgtccgagaagcaggtgaagatctggtttcagaaccgccgggtgaagcacaagaaagaaggcaaaggcagtaaccaccgcggcggagctggggcgggggccggcgggggcgcaccgcaaggctgcaagtgctcttcgctctcctcagccaaatgctcagaggacgacgacgaattgcccatgtctccatcttcctccgggaaggatgacagagatctcacagtcactccgtaggtgcgccttttagaggaccattggtttccccaccccccaccccgacccttcccgcacttcaaagactggtccccaggcacccgctggccaaccgacggatttcgttgggctttgcggtggtgcgcagctctaggcaaagctaagaccttagcagacacttgaagacagtgcccctgtcccttgggcttcagggtgtttaggaggactccaagcgatgaaggctgagtcctcctcctaggacacagcctcttctcccaggcacgcaggccggagcacagcgccttgctgaccgccagcgcctcttcgcctgccaactctgggctggttcaagcttcctcggttccactagtctctccttctcggtcaatctgggcttttcactccgcgagtggctgtttgcttttcttttaacatttctttcttgcccccaactcgtctccccccactgtggtcctttatgcaacacgtctatggacttaacttttccttccctcctcaggaagtctctcccttctgtccgtttgtcccgtcgctccaagggcgaattcacgatatcaagcggccgcaactcgagactctagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtggggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccgcggagatctaagctctagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttaaacctcgatcgagtctagatcaatatttcgagtacctctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgaatctagatcctctcttaagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 1777 bp, Rattus norvegicus
  source 1..1777: mol_type = other DNA
ccagcacctcgcgcgctccgggcggcgcccgcagcagcagccaaggtgattccagccccggcttgagccgcgcgtggagcctcccggacccgggaaactgcgggtggccgcggcagagggaggccactgcagaatccgcagttccctcgggcgccaggggcgggctggcagcaggtggaccgcgcgccggagccatgccgcgctctttcctggtggattcccttgtgctgcgggaagccagcgacaagaaggctccggagggcagcccgccaccgctcttcccctacgcggtccccccgccgcacgcgctccacggcctctcgccgggcgcctgccacgcgcgcaaggccggcttgctgtgcgtgtgtcccctctgtgtcaccgcttcgcagctgcacgggccccccgggccgccggcgctgccgctactcaaggcgtccttccctcccttcggatcgcagtactgccacgcacccctgggccgccagcactctgtgtctcccggagtcgcccacagcccggccgcggctgcagcagctgccgcactctaccagacctcctacccgctgccggatcccagacagtttcactgcatctccgtggacagcagctcgaaccagctgcccagcagtaagaggatgcggacggcgttcaccagcacgcagctcctggagctggagcgcgagttcgcctccaacatgtacctctcccgcctgcggcgcatcgagatcgcgacctatctgaatctgtccgagaagcaggtgaagatctggtttcagaaccgccgggtgaagcacaagaaagaaggcaagggcagtaaccaccgcggcggagctggggcgggggccggcgggggcgcaccgcaaggctgcaagtgctcttcgctctcctcagccaaatgctcagaggatgacgacgaattgcccatgtctccatcttcctccgggaaggatgacagagatctcacagtcactccgtaggtgcgccttttagaggaccattggtccccccccttccccggcccttcccacacttcaaagactggttcttaggctccggctgccaatcaatggatggggagggctttgcggtggtgcacagctctaggcagagctgagaccttagcagagacttgaagcccttgtcactagggcttcagggtgtttaggaggactccaaatggtgaacgctgggtcctcctcccaggacacagcttcttctccccccccaggcacgcaggccggagaacagcgccttgctgaccgccggcgcctcctcgccgctaactctggactggttcaggcttcctctatcccacaaccctctcttcctcggtcaagctgggctttttcactccgctagtggctatttgctttccttttaacattttttcttgttctcactcccccacagccccccccgtggtcgtttatgcaacgcgtttgttacccccccccccacacacacacacactccggaagcctcttccttctgtctgtttgtcccttaaacagggaccaggtcttgctgttcgaagaagtccccttagcagagaggactgtctcaaatggtattgtattgggggaaatgactgtttccaacactctccaccccctttcaaatgaagccgctgtaaacaacctctcccccatcgtccaggtcccgaccccttctggggactgactgactgtgttgttgtatggtctctgtaatgccagaagatatttatttatttatttatgtacaaaattttaaataaacttttttttcttagaaa

SEQ ID NO: 8 - DNA, 786 bp, Rattus norvegicus
  source 1..786: mol_type = other DNA
atgccgcgctctttcctggtggattcccttgtgctgcgggaagccagcgacaagaaggctccggagggcagcccgccaccgctcttcccctacgcggtccccccgccgcacgcgctccacggcctctcgccgggcgcctgccacgcgcgcaaggccggcttgctgtgcgtgtgtcccctctgtgtcaccgcttcgcagctgcacgggccccccgggccgccggcgctgccgctactcaaggcgtccttccctcccttcggatcgcagtactgccacgcacccctgggccgccagcactctgtgtctcccggagtcgcccacagcccggccgcggctgcagcagctgccgcactctaccagacctcctacccgctgccggatcccagacagtttcactgcatctccgtggacagcagctcgaaccagctgcccagcagtaagaggatgcggacggcgttcaccagcacgcagctcctggagctggagcgcgagttcgcctccaacatgtacctctcccgcctgcggcgcatcgagatcgcgacctatctgaatctgtccgagaagcaggtgaagatctggtttcagaaccgccgggtgaagcacaagaaagaaggcaagggcagtaaccaccgcggcggagctggggcgggggccggcgggggcgcaccgcaaggctgcaagtgctcttcgctctcctcagccaaatgctcagaggatgacgacgaattgcccatgtctccatcttcctccgggaaggatgacagagatctcacagtcactccgtag

SEQ ID NO: 9 - protein, 261 aa, Rattus norvegicus
MPRSFLVDSLVLREASDKKAPEGSPPPLFPYAVPPPHALHGLSPGACHARKAGLLCVCPLCVTASQLHGPPGPPALPLLKASFPPFGSQYCHAPLGRQHSVSPGVAHSPAAAAAAAALYQTSYPLPDPRQFHCISVDSSSNQLPSSKRMRTAFTSTQLLELEREFASNMYLSRLRRIEIATYLNLSEKQVKIWFQNRRVKHKKEGKGSNHRGGAGAGAGGGAPQGCKCSSLSSAKCSEDDDELPMSPSSSGKDDRDLTVTP